(19) United States
(12) Patent Application Publication
(10) Pub. No.: US 2026/0191863 A1
(43) Pub. Date: Jul. 9, 2026

(54) PYRROLOPYRIMIDINE COMPOSITIONS FOR TREATMENT OF ITK MEDIATED CONDITIONS

(71) Applicant: ACLARIS THERAPEUTICS, INC., Wayne, PA (US)

(72) Inventors: David Randolph ANDERSON, Salem, CT (US); Robert Alan ORTMANN, Edinburgh, IN (US); Wenjie LU, Wayne, PA (US)

(21) Appl. No.: 19/128,315

(22) PCT Filed: Nov. 7, 2023
(86) PCT No.: PCT/US2023/079020

§ 371 Date: May 8, 2025

Related U.S. Application Data
(60) Provisional application No. 63/382,835, filed on Nov. 8, 2022.
(60) Provisional application No. 63/538,913, filed on Sep. 18, 2023.

(51) Int. Cl. A61K 31/519; A61K 9/00

(52) U.S. Cl. CPC A61K 31/519; A61K 9/0053

(57) ABSTRACT

The present disclosure relates to oral compositions of Compound I or a derivative thereof. Several dosing ranges are disclosed along with SAD, MAD and PK data. Comparative data between Compound I, Tofacitinib, Ritlecitinib, and Cyclosporine A are also disclosed. Methods of use for treating an inflammatory condition are also disclosed.

### CROSS-REFERENCE TO RELATED APPLICATIONS

This application claims the benefit of U.S. Provisional Application No. 63/382,835 filed Nov. 8, 2022 and U.S. Provisional Application No. 63/538,913 filed Sep. 18, 2023. The disclosures of both of these applications are incorporated herein by reference. The disclosure of the application is incorporated herein by reference.

### SUMMARY

The present disclosure is directed to a method of treating an inflammatory condition in a human subject in need thereof, said method comprising orally administering to the human subject an oral dose selected from about 10 mg/day to about 80 mg/day of Compound I

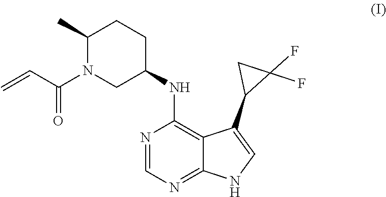 [SMILES: C=CC(=O)N1C[C@H](Nc2ncnc3[nH]cc([C@H]4CC4(F)F)c23)CC[C@@H]1C]

or a derivative thereof to treat said inflammatory condition.

The present disclosure is further directed to oral pharmaceutical compositions comprising Compound I or a derivative thereof, where the oral compositions comprise about 1 mg to about 100 mg of Compound I and a pharmaceutically acceptable carrier.

### BRIEF DESCRIPTION OF THE DRAWINGS

FIG. 1 depicts observed human Compound I PK (1-80 mg, Preliminary PK Parameters).

FIG. 2 depicts the food effect on AUC Data at 15 mg.

FIG. 3 depicts the formulation effect on AUC Data at 25 mg.

FIG. 4 depicts the αCD3/αCD28 stimulated IL2 mRNA production.

FIG. 5 depicts the αCD3/αCD28 stimulated IFNγ mRNA production.

FIG. 6 depicts the IL2 stimulated pSTAT5 in Lymphocytes.

FIG. 7 depicts the IL2 stimulated pSTAT5 in CD3+ T Cells.

FIG. 8 depicts the αCD3/IL15 stimulated IFNγ protein production.

FIGS. 9A-C depicts the SAD exposure response (9A), and SAD calculated response at Cₘₐₓ by dose (9B) for TCR (IL2 mRNA).

FIGS. 10A-C depicts the SAD exposure response (10A), and SAD calculated response at Cₘₐₓ by dose (10B) for ITK (IFNγ mRNA).

FIGS. 11A-C depicts the SAD exposure response (11A), and SAD calculated response at Cₘₐₓ by dose (11B) for JAK3 signaling (pSTAT5, lymph).

FIGS. 12A-C depicts the SAD exposure response (12A), and SAD calculated response at Cₘₐₓ by dose (12B) for JAK3 signaling (pSTAT5, CD3⁺ T).

FIGS. 13A-C depicts the SAD exposure response (13A), and SAD calculated response at Cₘₐₓ by dose (13B) for TCR & JAK signaling (IFNγ protein).

FIG. 14 depicts the doses for Compound I in cohorts 1-5 of example 2.

FIG. 15 depicts the pharmacokinetics of Compound I at several doses.

FIGS. 16A-C depicts αCD3 and αCD28 stimulated IL2 mRNA (16A), IL15 stimulated IFNγ production (16B), and αCD3 and αCD28 stimulated IFNγ production (7C16C) of BID cohorts.

FIGS. 17A-C depicts αCD3 and αCD28 stimulated IL2 mRNA (17A), IL15 stimulated IFNγ production (17B), and αCD3 and αCD28 stimulated IFNγ production (17C) of 30 mg QD vs 15 mg BID vs 10 mg TID cohorts.

FIGS. 18A-C depicts the Compound I MAD exploratory pharmacodynamics dose response data for all cohorts for αCD3 & αCD28 stimulated IL2mRNA (18A), IL15 stimulated IFNγ protein (18B), and αCD3 & IL15 stimulated IFNγ protein (18C).

FIG. 19 depicts the rat Antigen Induced Arthritis (AIA) Compound I QD vs BID dosing.

FIGS. 20A-B depicts simulated data for a 50 mg dose of ritlecitinib (11A) vs several Compound I doses (11B).

FIG. 21 depicts the percent change of initial body weight of mice in the mouse T cell transfer model.

FIGS. 22A-C depicts histopath score in the mouse T cell transfer model in the proximal colon (22A), distal colon (22B), and ileum (22C).

FIG. 23 depicts the mouse colitis model of Compound I vs. rilecitinib.

FIG. 24 depicts the mouse colitis model of Compound I vs. tofacitinib.

### DETAILED DESCRIPTION

### Definitions

Before the present compositions and methods are described, it is to be understood that this invention is not limited to the particular processes, formulations, compositions, or methodologies described, as these may vary. It is also to be understood that the terminology used in the description is for the purpose of describing the particular versions or embodiments only, and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments herein, the preferred methods, devices, and materials are now described. All publications mentioned herein are incorporated by reference in their entirety. Nothing herein is to be construed as an admission that embodiments herein are not entitled to antedate such disclosure by virtue of prior invention.

As used herein and in the appended claims, the singular forms “a,” “an,” and “the” include plural reference unless the context clearly dictates otherwise. Thus, for example, reference to an “ITK inhibitor” is a reference to one or more ITK inhibitors and equivalents thereof known to those skilled in the art, and so forth.

The transitional term “comprising,” which is synonymous with “including,” “containing,” or “characterized by,” is inclusive or open-ended and does not exclude additional, non-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. The compositions and methods of the present disclosure can comprise, consist essentially of, or consist of, the components or steps disclosed.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean plus or minus 10% of the numerical value of the number with which it is being used. Therefore, about 50 mg means in the range of 45 mg to 55 mg.

“Administering” when used in conjunction with a therapeutic means to administer a therapeutic to a patient whereby the therapeutic positively impacts the tissue to which it is targeted. Thus, as used herein, the term “administering”, when used in conjunction with an ITK inhibitor compound, can include, but is not limited to, providing an ITK inhibitor compound into or onto the target tissue; providing an ITK inhibitor compound systemically to a patient by, e.g., oral administration whereby the therapeutic reaches the target tissue.

As used herein, the term “a derivative thereof” refers to a salt thereof, a pharmaceutically acceptable salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, a geometric isomer thereof, a tautomer thereof, a mixture of tautomers thereof, an enantiomer thereof, a diastereomer thereof, a racemate thereof, a mixture of stereoisomers thereof, an isotope thereof (e.g., tritium, deuterium), or a combination thereof.

The term “substantially free” as used herein, alone or in combination, refers to the absence of isomers within the limits of detection of analytical methods such as nuclear magnetic resonance (NMR), gas chromatography/mass spectroscopy (GC/MS), high performance liquid chromatography (HPLC), or liquid chromatography/mass spectroscopy (LC/MS).

The term “condition” as used herein is intended to be generally synonymous, and is used interchangeably with, the terms “disorder,” “syndrome,” and “disease”, in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “ITK inhibitor” is used herein to refer to a compound that exhibits an IC₅₀ with respect to ITK activity of no more than about 100 M and more typically not more than about 50 μM, as measured in the ITK enzyme assay described generally herein. IC₅₀ is that concentration of inhibitor that reduces the activity of an enzyme (e.g., ITK) to half-maximal level. Certain compounds disclosed herein have been discovered to exhibit inhibition against ITK. In certain embodiments, compounds will exhibit an IC₅₀ with respect to ITK of no more than about 10 μM; in further embodiments, compounds will exhibit an IC₅₀ with respect to ITK of no more than about 5 μM; in yet further embodiments, compounds will exhibit an IC₅₀ with respect to ITK of not more than about 1 μM; in yet further embodiments, compounds will exhibit an IC₅₀ with respect to ITK of not more than about 200 nM, as measured in the ITK binding assay described herein.

As used herein, the term “pharmaceutically acceptable salt” refers to a salt prepared from a base or acid which is acceptable for administration to a patient, such as a mammal. The term “pharmaceutically acceptable salts” embraces salts commonly used to form alkali metal salts and to form addition salts of free acids or free bases. The nature of the salt is not critical, provided that it is pharmaceutically-acceptable. Such salts can be derived from pharmaceutically-acceptable inorganic or organic bases and from pharmaceutically-acceptable inorganic or organic acids.

Suitable pharmaceutically acceptable acid addition salts of the Compound I of embodiments herein may be prepared from an inorganic acid or an organic acid. All of these salts may be prepared by conventional means from the corresponding compound of embodiments herein by treating, e.g., the compound with the appropriate acid or base.

Pharmaceutically acceptable acids include both inorganic acids, for example hydrochloric, hydrobromic, hydroiodic, nitric, carbonic, sulfuric, phosphoric and diphosphoric acid; and organic acids, for example formic, acetic, trifluoroacetic, propionic, succinic, glycolic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, galacturonic, citric, fumaric, gluconic, glutamic, lactic, maleic, malic, mandelic, mucic, ascorbic, oxalic, pantothenic, succinic, tartaric, benzoic, acetic, xinafoic (1-hydroxy-2-naphthoic acid), napadisilic (1,5-naphthalenedisulfonic acid) and the like.

Salts derived from pharmaceutically-acceptable inorganic bases suitable for the formulations as described herein include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including alkyl amines, arylalkyl amines, heterocyclyl amines, cyclic amines, naturally-occurring amines and the like, such as arginine, betaine, caffeine, choline, chloroprocaine, diethanolamine, N-methylglucamine, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Other preferred salts according to embodiments herein are quaternary ammonium compounds wherein an equivalent of an anion (X−) is associated with the positive charge of the N atom. X⁻ may be an anion of various mineral acids (e.g., chloride, bromide, iodide, sulfate, nitrate, phosphate), or an anion of an organic acid (e.g. acetate, maleate, fumarate, citrate, oxalate, succinate, tartrate, malate, mandelate, trifluoroacetate, methanesulfonate, p-toluenesulfonate). X⁻ is preferably an anion selected from chloride, bromide, iodide, sulfate, nitrate, acetate, maleate, oxalate, succinate or trifluoroacetate. More preferably X⁻ is chloride, bromide, trifluoroacetate or methanesulfonate.

The Compound I of embodiments herein may exist in both non-solvated and solvated forms. The term solvate is used herein to describe a molecular complex comprising a compound of embodiments herein and an amount of one or more pharmaceutically acceptable solvent molecules. The term hydrate is employed when said solvent is water. Examples of solvate forms include, but are not limited to, Compound I of embodiments herein in association with water, acetone, dichloromethane, 2-propanol, ethanol, methanol, dimethyl sulfoxide (DMSO), ethyl acetate, acetic acid, ethanolamine, or mixtures thereof. It is specifically contemplated that in embodiments herein one solvent molecule can be associated with one molecule of the Compound I of embodiments herein, such as a hydrate.

In some embodiments herein one solvent molecule can be associated with one molecule of the compound described herein, such as a hydrate. In some embodiments, more than one solvent molecule may be associated with one molecule of the compound described herein, such as a dihydrate. Additionally, in some embodiments herein less than one solvent molecule may be associated with one molecule of the compound described herein, such as a hemihydrate. Furthermore, solvates of embodiments herein are contemplated as solvates of the compound described herein that retain the biological effectiveness of the non-solvate form of the compounds.

Embodiments herein also includes isotopically-labeled Compound I of embodiments herein, wherein one or more atoms is replaced by an atom having the same atomic number, but an atomic mass or mass number different from the atomic mass or mass number usually found in nature. Examples of isotopes suitable for inclusion in the Compound I of embodiments herein include isotopes of hydrogen, such as ²H and ³H carbon, such as ¹¹C, ³C and ¹⁴C, chlorine, such as ³¹Cl, fluorine, such as ⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labeled Compound I of embodiments herein, e.g., those incorporating a radioactive isotope, are useful in drug and/or substrate tissue distribution studies. The radioactive isotopes tritium, ³H, and carbon-14, ¹⁴C, are particularly useful for this purpose in view of their ease of incorporation and ready means of detection. Substitution with heavier isotopes such as deuterium, ²H may afford certain therapeutic advantages resulting from greater metabolic stability, e.g., increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled Compound I of embodiments herein can generally be prepared by conventional techniques known to those skilled in the art or by processes analogous to those described herein, using an appropriate isotopically-labeled reagent in place of the non-labeled reagent otherwise employed.

Preferred isotopically-labeled compounds include deuterated derivatives of the Compound I of embodiments herein. As used herein, the term deuterated derivative embraces Compound I of embodiments herein where in a particular position at least one hydrogen atom is replaced by deuterium. Deuterium (D or 2H) is a stable isotope of hydrogen which is present at a natural abundance of 0.015 molar %.

Hydrogen deuterium exchange (deuterium incorporation) is a chemical reaction in which a covalently bonded hydrogen atom is replaced by a deuterium atom. Said exchange (incorporation) reaction can be total or partial.

Typically, a deuterated derivative of a compound of embodiments herein has an isotopic enrichment factor (ratio between the isotopic abundance and the natural abundance of that isotope (the percentage of incorporation of deuterium at a given position in a molecule in the place of hydrogen) for each deuterium present at a site designated as a potential site of deuteration on the compound of at least 3500 (52.5% deuterium incorporation).

In some embodiments, the isotopic enrichment factor is at least 5000 (75% deuterium). In some embodiments, the isotopic enrichment factor is at least 6333.3 (95% deuterium incorporation). In some embodiments, the isotopic enrichment factor is at least 6633.3 (99.5% deuterium incorporation). It is understood that the isotopic enrichment factor of each deuterium present at a site designated as a site of deuteration is independent from the other deuteration sites.

The term “subject” as used herein and interchangeably with “patient”, includes, but is not limited to, humans and non-human vertebrates such as wild, domestic, and farm animals. In certain embodiments, the subject described herein is an animal. In certain embodiments, the subject is a mammal. In certain embodiments, the subject is a human. In certain embodiments, the subject is a non-human animal. In certain embodiments, the subject is a non-human mammal. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In another embodiment, the subject is a research animal such as a rodent, dog, or non-human primate. In certain embodiments, the subject is a non-human transgenic animal such as a transgenic mouse or transgenic pig.

The phrase “therapeutically effective” is intended to qualify the amount of active ingredients used in the treatment of a disease or disorder or on the effecting of a clinical endpoint.

The term “therapeutically acceptable” refers to those compounds, and a derivative thereof, which are suitable for use in contact with the tissues of patients without undue toxicity, irritation, and allergic response, are commensurate with a reasonable benefit/risk ratio, and are effective for their intended use.

The term “BID” as used herein refers to “bis in die” or “twice a day”.

The term “TID” as used herein refers to “ter in die” or “three times a day”.

The terms “treat,” “treated,” “treating”, or “treatment” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total, whether induction of or maintenance of), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease and prolonging disease-free survival as compared to disease-free survival if not receiving treatment and prolonging disease-free survival as compared to disease-free survival if not receiving treatment.

Embodiments herein are directed to oral pharmaceutical compositions that inhibit ITK activity and methods of treatment that involve administering to a human subject in need thereof an oral dose of the ITK inhibitor compound. Some embodiments include methods for the treatment of diseases in a human subject in need thereof that comprise orally administering the ITK inhibitor compound described herein.

The Tec (tyrosine kinase expressed in hepatocellular carcinoma) family of tyrosine kinases (TFTK) consists of five family members: Tec, BTK (Bruton's tyrosine kinase). BMX (bone marrow kinase on the X chromosome also known as ETK), RLK (resting lymphocyte kinase also known as TXK) and ITK (interleukin-2 inducible T cell kinase, also known as EMT and TSK). These kinases are central to the regulation of hematopoietic cell biology and more specifically the development and activity of lymphocytes and myeloid cells. The TFTK have structural similarities to other non-receptor tyrosine kinases while exhibiting some family specific motifs resulting in a diversity of domain structures associated with complex localization, scaffolding and activation mechanisms. Generally, TFTK contain an amino terminal plekstrin homology domain (PH domain) involved in lipid interactions and membrane targeting followed by a BTK homology domain (BH) that binds Zn²⁺ and an SH3 domain generally involved in proline rich domain binding. A phosphotyrosine binding SH2 domain and a carboxy terminal ATP binding kinase domain complete the TFTK structure. TFTK expression is generally limited to hematopoietic lineage cells with the exception of ETK and TEC that are expressed in the liver and endothelial cells, respectively. BMX is expressed in monocytes, granulocytes and cardiac endothelium while BTK is expressed in B cells and mast cells but not plasma cells and T cells. TEC, RLK and ITK are all expressed in T cells. To date the TFTK with the most clear biological role in T cells is ITK.

Antigen/MHC dependent activation of the T cell receptor (TCR) has been shown to transduce its signal through ITK. TCR stimulation results in the activation of the kinase LCK and subsequent phosphorylation of the immunoreceptor tyrosine-based activation motifs (ITAMs) on CD3 inducing the binding and activation of the kinase ZAP70. In turn, ZAP70 phosphorylates the adaptor proteins LAT and SLP-76, which together with LCK and other proteins forms a hetermultimeric signaling complex that activates PI3K and generates PIP₃ on the plasma membrane. ITK binds to this signaling complex via SH2 and SH3 domains and to PIP₃ through its PH domain, resulting in LCK dependent phosphorylation of ITK Y511 and subsequent ITK autophosphorylation of Y180. Activated ITK phosphorylates PLCγ1 that, once activated, hydrolyzes PIP₂ to the second messengers IP3 and DAG. The cellular consequences of these sequelae of events include calcium mobilization and flux, PKC and MEK/ERK pathway activation, and transcriptional activation via API, NF_{κactiv} NFAT. As a critical enzyme in the TCR activation pathway, ITK impacts T cell function in a number of ways including positive and negative selection, cellular differentiation, and cytokine production and release.

The role of ITK in T cell function has been delineated through genetic knockdown/kinase inactivation of the ITK gene in rodents and through characterizing human ITK mutant individuals. Mice with a null mutation of the itk gene expressed a decreased number of mature T cells and a block in thymocyte development as well as a decreased TCR driven T cell proliferative response. Interestingly IL2 and CD28 signaling as well as PMA/ionomycin driven responses remained unchanged, suggesting that the ITK response is membrane proximal and stimuli specific. It appears that ITK is responsible for amplification of TCR signaling versus an ‘on/off’ switch, as dual knockdown of the T cell expressing TFTK. ITK and RLK in mice produce a more complete TCR inactivation phenotype compared with ITK genetic deletion alone. In contrast to the modulatory effect that ITK appears to have on naïve T cell activation, it plays a more significant role in T helper cell differentiation. Several studies in ITK deficient mice have demonstrated a reduction in the Th2 protective response to parasitic infection. This reduced Th2 response was linked to a decrease in concentrations of Th2 cytokines IL4, IL5, IL13 and IL10 and to a reduction in RLK expression. In contrast to the ITK requirement for mounting Th2 driven responses, its impact on Th1 responses is modest. For example, IFNγ production in ITK knockout cells is partially inhibited while the double ITK/RLK knockout has a more severe phenotype. Evaluation of Th17 T helper cells in ITK knockout in vivo and in vitro studies demonstrated a reduction of IL17A mRNA and protein while having little impact on IL17F. The role of ITK in cytotoxic CD8+ T cells was investigated using ITK knockout mice. Stimulation of CD8+ T cells deficient in ITK results in a reduction in activation of PLCg1, ERK and p38 MAPK and loss of Ca²⁺ response resulting in decreased proliferative response and effector cytokine production (IL2. IL4 and IFNγ) while not impacting cytolytic capacity of these cells. In addition to the defects observed in CD4+ and CD8+ T cells, natural killer T cell development and TCR stimulated response is reduced in ITK knockout cells and animals.

Rodent genetic knockout studies reflect the impact of enzyme expression, not necessarily its catalytic activity, on biological responses. ITK, through its multiple domain structure, has a role in scaffolding, in addition to its catalytic role. It is important to delineate the impact of blocking each of these functions on cellular biology. Kinase activity-independent ITK activities include recruitment of the guanine nucleotide exchange factor VAV to the cell membrane associated with actin polymerization (PH and SH2 domain dependent), antigen receptor stimulation, and receptor activation of SRF. However, ITK knockout mice expressing an ITK kinase domain deleted transgene demonstrated that the kinase domain is essential for induction of a normal Th2 response.

The relationship between ITK expression and activity and human disease has recently been documented in studies characterizing individuals exhibiting mutations in the gene encoding this protein and or correlation between expression and disease. The ITK gene was found to be elevated in peripheral blood T cells from patients with moderate to severe atopic dermatitis, a Th2 driven chronic inflammatory skin disease. An investigation of disease-associated single nucleotide polymorphisms (SNP) in seasonal allergic rhinitis identified ITK as a significant risk factor. A human primary immunodeficiency was uncovered in siblings that died from immune dysregulation resulting in lymphoproliferation following Epstein Barr Virus (EBV) infection. This disorder was linked to a missense (R335W) mutation in the SH2 domain of ITK resulting in structural instability and reduced steady state levels of the enzyme. The finding was confirmed and extended in studies that identified three patients harboring a C1764G nonsense mutation in ITK resulting in a premature stop codon and reduced expression and/or activity of the protein. These patients presented with EBV-positive Hodgkins Lymphoma. These two reports suggest that mutational disruption of the ITK gene in humans results in an autosomal recessive lymphoproliferative disorder and identifies this kinase as a critical modulator in T cell biology.

In addition to the human genetic data summarized above, animal models support ITK as a therapeutic target for autoimmune and inflammatory disease. ITK knockout mice demonstrate reduced airway hypersensitivity and inflammation in models of allergic asthma. In a murine model of atopic dermatitis, ITK deficient mice do not develop inflammation while ITK inhibition reduces the response in wild type mice. The ITK dependent regulation of TCR dependent Ca²⁺ mobilization and transcription factor induction makes it a critical factor in protecting against Influenza A and HIV infection and viral replication. ITK inhibitors have been shown to alter HIV replication at multiple stages and have the potential as effective HIV therapeutics.

From an oncology perspective, studies have demonstrated that ITK inhibitors selectively target the killing of acute lymphoblastic T-cell leukemia and cutaneous T-cell lymphoma while normal T cells are minimally impacted. ITK is highly expressed in transformed T-cell lines relative to normal T cells and other cancer cell lines. The impact of ITK inhibition on T cell tumors was confirmed in mouse xenograph models. Cancer evasion of the immune system as a result of tumor antigen tolerance induction versus priming is critical for tumor survival. Tumors that develop a microenvironment that induces T cell unresponsiveness demonstrate altered T cell gene expression suggesting skewing to the Th2 phenotype. ITK inhibition will favor Th1 differentiation and could be used to enhance cancer immunotherapy.

The utility of simultaneously inhibiting signals downstream of both the T cell receptor and cytokines receptors is best exemplified by the field of organ transplantation (Halloran). Standard-of-care in the immediate post-transplant setting is to prevent immunological graft rejection by dosing with a cocktail of inhibitors that impinge on lymphocyte activation at different points. The most common drugs used in both the initial post-transplant period, as well as the discharge setting—are a calcineurin inhibitor such as Prograf or Neoral, which block TCR signaling, and an anti-metabolite, such mycophenolate mofetil or rapamycin, which block the proliferation induced downstream of cytokines such as IL-2 or IL-15. These drugs cannot be used as monotherapy, as it would require higher doses resulting in drug-specific toxicity. By using these drugs at sub-optimal doses simultaneously, synergistic immunosuppression is achieved with reduced toxicity. ITK is downstream of TCR and JAK3 is downstream of the common g chain of cytokine receptors. By virtue of covalently modifying the cysteine in the catalytic domain of both kinases, it is possible to inhibit both enzymes with a single drug and thereby synergistically inhibit the activation of T cells.

Also provided are embodiments wherein any embodiment described herein may be combined with any one or more of these embodiments, provided the combination is not mutually exclusive.

### Oral Compositions

Embodiments herein are directed to pharmaceutical compositions formulated for oral administration (“oral pharmaceutical composition”), comprising about 1 mg to about 100 mg of Compound I as shown below

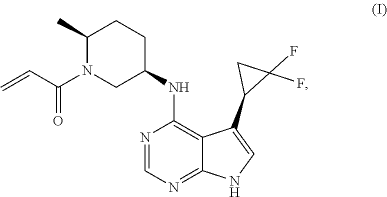 [SMILES: C=CC(=O)N1C[C@H](Nc2ncnc3[nH]cc([C@H]4CC4(F)F)c23)CC[C@@H]1C]

or a derivative thereof, and a pharmaceutically acceptable carrier.

Compound I is also referred to herein by its chemical name, i.e., 1-((2S,5R)-5-((5-((R)-2,2-difluorocyclopropyl)-7H-pyrrolo[2,3-d]pyrimidin-4-yl)amino)-2-methylpiperidin-1-yl)prop-2-en-1-one.

Compound I may be prepared according to the methods described in U.S. Pat. No. 11,021,482, which is hereby incorporated by reference in its entirety.

In any embodiment, the oral pharmaceutical composition disclosed herein comprises 1 mg of Compound I. In any embodiment, the oral pharmaceutical composition disclosed herein comprises 3 mg of Compound I. In any embodiment, the oral pharmaceutical composition disclosed herein comprises 5 mg of Compound I. In any embodiment, the oral pharmaceutical composition disclosed herein comprises 10 mg of Compound I. In any embodiment, the oral pharmaceutical composition disclosed herein comprises 15 mg of Compound I. In any embodiment, the oral pharmaceutical composition disclosed herein comprises 25 mg of Compound I. In any embodiment, the oral pharmaceutical composition disclosed herein comprises 30 mg of Compound I. In any embodiment, the oral pharmaceutical composition disclosed herein comprises 40 mg of Compound I. In any embodiment, the oral pharmaceutical composition disclosed herein comprises 50 mg of Compound I. In any embodiment, the oral pharmaceutical composition disclosed herein comprises 80 mg of Compound I.

In any embodiment, the oral pharmaceutical compositions described herein comprise Compound I in an amount of about 1 mg to about 100 mg, or any amount in between. In an embodiment, Compound I is present in the pharmaceutical composition as described herein in an amount of about 2 mg to about 100 mg. In an embodiment, Compound I is present in the pharmaceutical composition about 3 mg to about 100 mg. In an embodiment. Compound I is present in the pharmaceutical composition about 4 mg to about 100 mg. In an embodiment, Compound I is present in the pharmaceutical composition about 5 mg to about 100 mg. In an embodiment, Compound I is present in the pharmaceutical composition about 10 mg to about 100 mg. In an embodiment, Compound I is present in the pharmaceutical composition about 15 mg to about 100 mg. In an embodiment, Compound I is present in the pharmaceutical composition about 20 mg to about 100 mg. In an embodiment, Compound I is present in the pharmaceutical composition about 25 mg to about 100 mg. In an embodiment, Compound I is present in the pharmaceutical composition about 30 mg to about 100 mg. In an embodiment, Compound I is present in the pharmaceutical composition about 35 mg to about 100 mg. In an embodiment, Compound I is present in the pharmaceutical composition about 40 mg to about 100 mg. In an embodiment, Compound I is present in the pharmaceutical composition about 45 mg to about 100 mg. In an embodiment, Compound I is present in the pharmaceutical composition about 50 mg to about 100 mg. In an embodiment, Compound I is present in the pharmaceutical composition about 1 mg to about 90 mg. In an embodiment, Compound I is present in the pharmaceutical composition about 1 mg to about 80 mg. In an embodiment, Compound I is present in the pharmaceutical composition about 1 mg to about 70 mg. In an embodiment, Compound I is present in the pharmaceutical composition about 1 mg to about 60 mg. In an embodiment, Compound I is present in the pharmaceutical composition about 1 mg to about 50 mg. In an embodiment, Compound I is present in the pharmaceutical composition about 1 mg to about 40 mg. In an embodiment, Compound I is present in the pharmaceutical composition about 1 mg to about 30 mg. In an embodiment, Compound I is present in the pharmaceutical composition about 1 mg to about 20 mg. In an embodiment, Compound I is present in the pharmaceutical composition about 1 mg to about 10 mg. In an embodiment, the oral pharmaceutical compositions described herein comprise Compound I in an amount of about 10 mg to about 80 mg. In an embodiment, the oral pharmaceutical compositions described herein comprise Compound I in an amount of about 5 mg to about 40 mg.

In an embodiment, Compound I is present in the pharmaceutical composition as described herein in an amount of about 0.1 mg. In an embodiment, Compound I is present in the pharmaceutical composition as described herein in an amount of about 0.5 mg. In an embodiment, Compound I is present in the pharmaceutical composition as described herein in an amount of about 1 mg. In an embodiment. Compound I is present in the pharmaceutical composition as described herein in an amount of about 2 mg. In an embodiment, Compound I is present in the pharmaceutical composition as described herein in an amount of about 3 mg. In an embodiment, Compound I is present in the pharmaceutical composition as described herein in an amount of about 4 mg. In an embodiment. Compound I is present in the pharmaceutical composition as described herein in an amount of about 5 mg. In an embodiment, Compound I is present in the pharmaceutical composition as described herein in an amount of about 6 mg. In an embodiment. Compound I is present in the pharmaceutical composition as described herein in an amount of about 7 mg. In an embodiment, Compound I is present in the pharmaceutical composition as described herein in an amount of about 8 mg. In an embodiment, Compound I is present in the pharmaceutical composition as described herein in an amount of about 9 mg. In an embodiment. Compound I is present in the pharmaceutical composition as described herein in an amount of about 10 mg. In an embodiment, Compound I is present in the pharmaceutical composition as described herein in an amount of about 11 mg. In an embodiment, Compound I is present in the pharmaceutical composition as described herein in an amount of about 12 mg. In an embodiment, Compound I is present in the pharmaceutical composition as described herein in an amount of about 13 mg. In an embodiment, Compound I is present in the pharmaceutical composition as described herein in an amount of about 14 mg. In an embodiment. Compound I is present in the pharmaceutical composition as described herein in an amount of about 15 mg. In an embodiment, Compound I is present in the pharmaceutical composition as described herein in an amount of about 16 mg. In an embodiment, Compound I is present in the pharmaceutical composition as described herein in an amount of about 17 mg. In an embodiment, Compound I is present in the pharmaceutical composition as described herein in an amount of about 18 mg. In an embodiment, Compound I is present in the pharmaceutical composition as described herein in an amount of about 19 mg. In an embodiment. Compound I is present in the pharmaceutical composition as described herein in an amount of about 20 mg. In an embodiment, Compound I is present in the pharmaceutical composition as described herein in an amount of about 21 mg. In an embodiment, Compound I is present in the pharmaceutical composition as described herein in an amount of about 22 mg. In an embodiment, Compound I is present in the pharmaceutical composition as described herein in an amount of about 23 mg. In an embodiment, Compound I is present in the pharmaceutical composition as described herein in an amount of about 24 mg. In an embodiment, Compound I is present in the pharmaceutical composition as described herein in an amount of about 25 mg. In an embodiment, Compound I is present in the pharmaceutical composition as described herein in an amount of about 26 mg. In an embodiment, Compound I is present in the pharmaceutical composition as described herein in an amount of about 27 mg. In an embodiment, Compound I is present in the pharmaceutical composition as described herein in an amount of about 28 mg. In an embodiment, Compound I is present in the pharmaceutical composition as described herein in an amount of about 29 mg. In an embodiment, Compound I is present in the pharmaceutical composition as described herein in an amount of about 30 mg. In an embodiment, Compound I is present in the pharmaceutical composition as described herein in an amount of about 31 mg. In an embodiment, Compound I is present in the pharmaceutical composition as described herein in an amount of about 32 mg. In an embodiment, Compound I is present in the pharmaceutical composition as described herein in an amount of about 33 mg. In an embodiment, Compound I is present in the pharmaceutical composition as described herein in an amount of about 34 mg. In an embodiment, Compound I is present in the pharmaceutical composition as described herein in an amount of about 35 mg. In an embodiment, Compound I is present in the pharmaceutical composition as described herein in an amount of about 36 mg. In an embodiment, Compound I is present in the pharmaceutical composition as described herein in an amount of about 37 mg. In an embodiment, Compound I is present in the pharmaceutical composition as described herein in an amount of about 38 mg. In an embodiment, Compound I is present in the pharmaceutical composition as described herein in an amount of about 39 mg. In an embodiment, Compound I is present in the pharmaceutical composition as described herein in an amount of about 40 mg. In an embodiment, Compound I is present in the pharmaceutical composition as described herein in an amount of about 41 mg. In an embodiment, Compound I is present in the pharmaceutical composition as described herein in an amount of about 42 mg. In an embodiment, Compound I is present in the pharmaceutical composition as described herein in an amount of about 43 mg. In an embodiment, Compound I is present in the pharmaceutical composition as described herein in an amount of about 44 mg. In an embodiment, Compound I is present in the pharmaceutical composition as described herein in an amount of about 45 mg. In an embodiment, Compound I is present in the pharmaceutical composition as described herein in an amount of about 46 mg. In an embodiment, Compound I is present in the pharmaceutical composition as described herein in an amount of about 47 mg. In an embodiment, Compound I is present in the pharmaceutical composition as described herein in an amount of about 48 mg. In an embodiment, Compound I is present in the pharmaceutical composition as described herein in an amount of about 49 mg. In an embodiment, Compound I is present in the pharmaceutical composition as described herein in an amount of about 50 mg. In an embodiment, Compound I is present in the pharmaceutical composition as described herein in an amount of about 51 mg. In an embodiment, Compound I is present in the pharmaceutical composition as described herein in an amount of about 52 mg. In an embodiment, Compound I is present in the pharmaceutical composition as described herein in an amount of about 53 mg. In an embodiment, Compound I is present in the pharmaceutical composition as described herein in an amount of about 54 mg. In an embodiment, Compound I is present in the pharmaceutical composition as described herein in an amount of about 55 mg. In an embodiment, Compound I is present in the pharmaceutical composition as described herein in an amount of about 56 mg. In an embodiment, Compound I is present in the pharmaceutical composition as described herein in an amount of about 57 mg. In an embodiment, Compound I is present in the pharmaceutical composition as described herein in an amount of about 58 mg. In an embodiment, Compound I is present in the pharmaceutical composition as described herein in an amount of about 59 mg. In an embodiment, Compound I is present in the pharmaceutical composition as described herein in an amount of about 60 mg. In an embodiment, Compound I is present in the pharmaceutical composition as described herein in an amount of about 61 mg. In an embodiment, Compound I is present in the pharmaceutical composition as described herein in an amount of about 62 mg. In an embodiment, Compound I is present in the pharmaceutical composition as described herein in an amount of about 63 mg. In an embodiment, Compound I is present in the pharmaceutical composition as described herein in an amount of about 64 mg. In an embodiment, Compound I is present in the pharmaceutical composition as described herein in an amount of about 65 mg. In an embodiment, Compound I is present in the pharmaceutical composition as described herein in an amount of about 66 mg. In an embodiment, Compound I is present in the pharmaceutical composition as described herein in an amount of about 67 mg. In an embodiment, Compound I is present in the pharmaceutical composition as described herein in an amount of about 68 mg. In an embodiment, Compound I is present in the pharmaceutical composition as described herein in an amount of about 69 mg. In an embodiment. Compound I is present in the pharmaceutical composition as described herein in an amount of about 70 mg. In an embodiment, Compound I is present in the pharmaceutical composition as described herein in an amount of about 71 mg. In an embodiment, Compound I is present in the pharmaceutical composition as described herein in an amount of about 72 mg. In an embodiment, Compound I is present in the pharmaceutical composition as described herein in an amount of about 73 mg. In an embodiment, Compound I is present in the pharmaceutical composition as described herein in an amount of about 74 mg. In an embodiment, Compound I is present in the pharmaceutical composition as described herein in an amount of about 75 mg. In an embodiment, Compound I is present in the pharmaceutical composition as described herein in an amount of about 76 mg. In an embodiment, Compound I is present in the pharmaceutical composition as described herein in an amount of about 77 mg. In an embodiment, Compound I is present in the pharmaceutical composition as described herein in an amount of about 78 mg. In an embodiment, Compound I is present in the pharmaceutical composition as described herein in an amount of about 79 mg. In an embodiment, Compound I is present in the pharmaceutical composition as described herein in an amount of about 80 mg. In an embodiment, Compound I is present in the pharmaceutical composition as described herein in an amount of about 81 mg. In an embodiment, Compound I is present in the pharmaceutical composition as described herein in an amount of about 82 mg. In an embodiment, Compound I is present in the pharmaceutical composition as described herein in an amount of about 83 mg. In an embodiment, Compound I is present in the pharmaceutical composition as described herein in an amount of about 84 mg. In an embodiment, Compound I is present in the pharmaceutical composition as described herein in an amount of about 85 mg. In an embodiment, Compound I is present in the pharmaceutical composition as described herein in an amount of about 86 mg. In an embodiment, Compound I is present in the pharmaceutical composition as described herein in an amount of about 87 mg. In an embodiment, Compound I is present in the pharmaceutical composition as described herein in an amount of about 88 mg. In an embodiment, Compound I is present in the pharmaceutical composition as described herein in an amount of about 89 mg. In an embodiment, Compound I is present in the pharmaceutical composition as described herein in an amount of about 90 mg. In an embodiment, Compound I is present in the pharmaceutical composition as described herein in an amount of about 91 mg. In an embodiment, Compound I is present in the pharmaceutical composition as described herein in an amount of about 92 mg. In an embodiment, Compound I is present in the pharmaceutical composition as described herein in an amount of about 93 mg. In an embodiment. Compound I is present in the pharmaceutical composition as described herein in an amount of about 94 mg. In an embodiment, Compound I is present in the pharmaceutical composition as described herein in an amount of about 95 mg. In an embodiment, Compound I is present in the pharmaceutical composition as described herein in an amount of about 96 mg. In an embodiment, Compound I is present in the pharmaceutical composition as described herein in an amount of about 97 mg. In an embodiment, Compound I is present in the pharmaceutical composition as described herein in an amount of about 98 mg. In an embodiment. Compound I is present in the pharmaceutical composition as described herein in an amount of about 99 mg. In an embodiment, Compound I is present in the pharmaceutical composition as described herein in an amount of about 100 mg. In preferred embodiments, Compound I is present in the pharmaceutical composition as described herein in an amount of about 1 mg, about 3 mg, about 5 mg, about 10 mg, about 15 mg, about 25 mg, about 30 mg, about 40 mg, about 50 mg, or about 80 mg.

In any embodiment, Compound I of the oral composition as disclosed herein comprises a free base. In any embodiment, Compound I of the oral composition as disclosed herein comprises a pharmaceutically acceptable salt.

In any embodiment, the pharmaceutically acceptable salt is an acid addition salt. Suitable acid addition salts include those formed with both organic and inorganic acids. Pharmaceutically acceptable acids include both inorganic acids, for example hydrochloric, hydrobromic, hydroiodic, nitric, carbonic, sulfuric, phosphoric and diphosphoric acid; and organic acids, for example formic, acetic, trifluoroacetic, propionic, succinic, glycolic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, galacturonic, citric, fumaric, gluconic, glutamic, lactic, maleic, malic, mandelic, mucic, ascorbic, oxalic, pantothenic, succinic, tartaric, benzoic, acetic, xinafoic (1-hydroxy-2-naphthoic acid), napadisilic (1,5-naphthalenedisulfonic acid) and the like.

In any embodiment, the pharmaceutically acceptable salt is a basic addition salt. Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, NN-dimethylaniline. N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

In any embodiment, Compound I of the oral compositions is a non-solvated form or in a solvated form. In any embodiment herein one solvent molecule can be associated with one molecule of Compound I as described herein, such as a hydrate. In some embodiments, more than one solvent molecule may be associated with one molecule of Compound I as described herein, such as a dihydrate. Additionally, in some embodiments herein less than one solvent molecule may be associated with one molecule of Compound I as described herein, such as a hemihydrate. Furthermore, solvates of embodiments herein are contemplated as solvates of Compound I as described herein that retain the biological effectiveness of the non-solvate form of Compound I.

In any embodiment, Compound I of the oral composition is a deuterated derivative. As used herein, the term deuterated derivative embraces Compound I of embodiments herein where in a particular position at least one hydrogen atom is replaced by deuterium. Deuterium (D or ²H) is a stable isotope of hydrogen which is present at a natural abundance of 0.015 molar %.

In any embodiment, the oral composition of the present disclosure comprises Compound I as disclosed herein formulated by admixture with a pharmaceutically acceptable carrier or excipient. In certain embodiments, the pharmaceutical compositions include the therapeutically effective amount of Compound I and a physiologically acceptable diluent or carrier. In certain embodiments, the pharmaceutical composition further includes one or more additional therapeutic components and/or adjuvants.

In any embodiment, the oral compositions disclosed herein may further comprise pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for preparation and administration are known in the art and an artisan can refer to various pharmacologic references for guidance. For example, Banker, G. S., & Rhodes, C. T. (2002). Modern pharmaceutics. New York: Marcel Dekker; and Goodman, L. S., Brunton, L. L., Chabner, B., & Knollmann. B. C. (2011). Goodman & Gilman's pharmacological basis of therapeutics. New York: McGraw-Hill, can be consulted.

The oral pharmaceutical compositions as disclosed herein can be formulated readily by combining Compound I with pharmaceutically acceptable carriers well known in the art. Such carriers enable the Compound I of embodiments herein to be formulated as nanoparticles, nanoparticle suspension, tablets, troches, pills, dragees, capsules, powders, liquids, gels, syrups, slurries, suspensions and the like, for oral ingestion by a subject to be treated. Oral compositions can also be prepared using a fluid carrier for use as a mouthwash, wherein the compound in the fluid carrier is applied orally and swished and expectorated or swallowed. Pharmaceutical preparations for oral administration can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropyl methylcellulose (HPMC), sodium carboxymethylcellulose (CMC), and polyvinylpyrrolidone (PVP). If desired, disintegrating agents can be added, such as, but not limited to, the cross-linked PVP, agar, or alginic acid or a salt thereof such as sodium alginate.

Dragee cores can be provided with suitable coatings. For this purpose, concentrated sugar solutions can be used, which can optionally contain gum arabic, talc, PVP, carbopol gel, polyethylene glycol (PEG), and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredient in an admixture with one or more fillers (e.g., lactose), one or more binders (e.g., starches), and/or one or more lubricants (e.g., talc or magnesium stearate) and, optionally, one or more stabilizers. In soft capsules, the active compound can be dissolved or suspended in suitable liquids, such as fatty oils, liquid paraffin, or liquid PEG. In addition, stabilizers can be added. All compositions for oral administration should be in dosages (e.g., about 1 mg to about 100 mg) suitable for such administration.

In some embodiments, the oral compositions may take the form of, e.g., lozenges, tablets or capsules prepared by conventional means with pharmaceutically acceptable excipients such as binding agents (e.g., pregelatinized maize starch, PVP or HPMC); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, e.g., sugars, films or enteric coatings. Additionally, the pharmaceutical compositions containing Compound I as disclosed herein can be in any form suitable for oral use, including, e.g., troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

In one embodiment, the oral pharmaceutical composition as disclosed herein is a tablet. Tablets may contain Compound I in admixture with non-toxic pharmaceutically acceptable excipients which are suitable for the manufacture of tablets. These excipients may be e.g., inert diluents, such as calcium carbonate, sodium carbonate, lactose, calcium phosphate or sodium phosphate; granulating and disintegrating agents (e.g., corn starch, or alginic acid): binding agents (for example starch, gelatin or acacia); and lubricating agents (for example magnesium stearate, stearic acid or talc). The tablets may be uncoated or they may be coated by known techniques to delay disintegration and absorption in the gastrointestinal tract and thereby provide a sustained action over a longer period. In some embodiments, the tablet is formulated for immediate release. In some embodiments, the tablet is formulated for controlled release. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release. The pharmaceutical compositions described herein may also be in the form of oil-in-water emulsions.

In some embodiments, the oral composition comprises Compound I and a buffer. In some embodiments, the buffer may be selected from the group consisting of citric acid monohydrate, sodium phosphate, water, and a combination thereof. In some embodiments, the oral composition comprises Compound I and a stabilizer. In some embodiments, the stabilizer is selected from a group consisting of povidone, sodium benzoate, water, sodium lauryl sulfate, and a combination thereof. In some embodiments, the oral composition further includes a buffer, an acid, sodium benzoate, sodium phosphate, citric acid, or a combination thereof. In some embodiments, the oral composition comprises Compound I and a stabilizer and a buffer. In some embodiments, the oral composition further comprises a lubricant, a pH modifier, a binder, a diluent, a granulating agent, a glidant, a disintegrant, a filler, a sorbent, an anti-adherent, a coloring agent, a compression aid, a coating material, a sweetener, a preservative, an antioxidant, or a combination thereof. In some embodiments, Compound I is in a therapeutically effective amount (e.g., about 5 mg to about 100 mg). In some embodiments, the oral composition is a suspension, tablet, capsule, nanoparticle powder, nanoparticle suspension, cachet, pellet, pill, powder, granules, or a combination thereof.

In some embodiments, the lubricant may be selected from the group consisting of stearic acid or its salts (e.g., magnesium stearate, calcium stearate), sodium lauryl sulfate, PEG, mineral oil, sodium benzoate, glyceryl palmitostearate, glyceryl behenate, sodium stearyl fumarate, and a combination thereof.

In some embodiments, the pH modifier may be an acid (e.g., hydrochloric acid, acetic acid, citric acid, phosphoric acid, sulfuric acid, or a combination thereof).

In some embodiments, the binder may be selected from the group consisting of a natural or synthetic polymer (e.g., starches, sugars, sugar alcohols, or cellulose derivatives) such as gelatin, glucose, lactose, sorbitol, xylitol, maltitol, methyl cellulose, microcrystalline cellulose (MCC), ethyl cellulose, HPMC, hydroxypropyl cellulose (HPC), starch, PVP, PEG, sodium alginate, CMC, and a combination thereof.

In some embodiments, the compression aid may be selected from the group consisting of silicified microcrystalline cellulose, microcrystalline cellulose, a physical mixture of MCC-colloidal silicon dioxide, and a combination thereof.

In some embodiments, the disintegrant may be selected from the group consisting of starch, cellulose derivatives and alginates, PVP, croscarmellose sodium, sodium starch glycolate, and a combination thereof.

In some embodiments, the filler may be selected from the group consisting of lactose, sucrose, glucose, mannitol, sorbitol, calcium carbonate, magnesium stearate, plant cellulose, dibasic calcium phosphate, dibasic sodium phosphate, vegetable fats and oils, and a combination thereof.

In some embodiments, the diluent may be selected from the group consisting of sugar compounds (e.g., sucrose, lactose, dextrin, glucose, sorbitol, or the like), inorganic compounds (e.g., silicates, calcium salts, or magnesium salts), sodium chloride, potassium chloride, and a combination thereof.

In some embodiments, the preservative may be selected from the group consisting of an antioxidant (e.g., vitamin A, vitamin E, vitamin C, retinyl palmitate, and selenium), an amino acid (e.g., cysteine, or methionine), citric acid, sodium citrate, a synthetic preservative (e.g., a paraben such as methyl paraben or propyl paraben), and a combination thereof.

In some embodiments, the glidant may be selected from the group consisting of colloidal anhydrous silicon and other silica compounds, such as fumed silica, magnesium carbonate, colloidal silicon dioxide (AEROSIL®), cornstarch, talc, and a combination thereof.

In some embodiments, the oral composition comprising Compound I is a capsule. In some embodiments, the capsule comprises an inner coating made from a high fat emulsion. In some embodiments, the capsule comprises a high fat coating that is either on the inside or the outside of the capsule. In some embodiments, the capsule comprises HPMC. In some embodiments, the capsule may be a HPMC capsule. In some embodiments, the capsule may be enteric coated. In some embodiments, the capsule may be a silica capsule, such as silica sold under the trade name SYLOID®. In some embodiments, the capsule comprises cyclodextrin. In some embodiments, the capsule may be a cyclodextrin complex enteric capsule.

In some embodiments, the oral formulation comprising Compound I is a tablet. In some embodiments, the tablet contains Compound I in the form of nanoparticles. In some embodiments, the tablet may be coated. In some embodiments, the tablet may be coated with an enteric coating. In some embodiments, the tablet may be coated with a coating selected from a sugar coating, film coating, organic film coating, aqueous film coating, pan coating, dip coating, electrostatic coating, compression coating, plasticizer dry coating, heat dry coating, electrostatic dry coating, or the like. Some ingredients used for coating may include aqueous acrylic enteric system such as that sold under the trade name ACRYL-EZE®, film coating system sold under the trade name OPADRY®, HPMC, methyl hydroxyethyl cellulose, ethylcellulose, povidone, cellulose acetate phthalate, acrylate polymers (such as those sold under the trade name EUDRAGIT® L & EUDRAGIT® S), HPMC phthalate, or a combination thereof.

In some embodiments, the oral composition comprises Compound I in the form of a nanoparticle suspension (nanosuspension). In some embodiments, the nanosuspension comprises Compound I, a stabilizer, and a buffer. In some embodiments, the nanosuspension may further comprise a pH modifier. In some embodiments, the pH modifier may be selected from a group consisting of hydrochloric acid, acetic acid, citric acid, phosphoric acid, sulfuric acid, and a combination thereof. In some embodiments, the pH modifier may be hydrochloric acid. In some embodiments, the hydrochloric acid may be 1.0N hydrochloric acid. In some embodiments, the stabilizer may be selected from the group consisting of povidone, sodium lauryl sulfate, sodium benzoate, WFI quality water such as that sold under the trade name HYCLONE™, or a combination thereof. In some embodiments, the buffer solution may include WFI quality water, sodium phosphate (dibasic. 7-hydrate, crystal), citric acid monohydrate, or a combination thereof.

The nanoparticle suspension may be manufactured by suspending particles of the active in the excipients, reducing particles to the desired particle size using grinding media in a mill, and then diluting the suspension to the final volume. In some embodiments, the grinding media used may be selected from ceramic, agate, silicon nitride, sintered corundum, zirconia, stainless steel, chrome steel, Cr—Ni steel, tungsten carbide, glass (yttrium-stabilized), cross-linked polystyrene resins, plastic polyamide, pearls, or a combination thereof. In some embodiments, the mill may be a stationary agitated vessel or a recirculating mill.

A tablet may be manufactured by spraying the nanosuspension (above) onto sucrose to form a spray granulate intermediate, granulating the spray granulate intermediate with excipients to form a final granulation, and compressing the final granulation to form a tablet. The sucrose could be any sugar, including, e.g., glucose, fructose, maltose, galactose, lactose, or the like. In some embodiments, the excipients for the tablet formulation may include lactose monohydrate, PVP, silicified microcrystalline cellulose (e.g., sold under the trade name PROSOLV® SMCC HD 90), magnesium stearate, or a combination thereof. In some embodiments, the tablet comprises about 1 mg to about 100 mg of Compound I.

In some embodiments the oral composition as described herein is a dry powder. In some embodiments, the dry powder may be encapsulated or made into a suspension. In some embodiments, the suspension may be a nanoparticle suspension or a milled suspension.

In some embodiments, liquid preparations for oral administration may take the form of, e.g., elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, CREMOPHORE® or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers (e.g., PEG).

The compositions can further include one or more additional pharmaceutical agents such as an anti-inflammatory drug, an anti-atherosclerotic drug, an immunosuppressive drug, an immunomodulatory drug, a cytostatic drug, an angiogenesis inhibitor, a kinase inhibitor, a cytokine blocker, and an inhibitor of cell adhesion molecules.

### Methods of Use

Some embodiments herein are directed to a method of modulation of an ITK-mediated function in a human subject comprising administering an oral dose of about 10 mg/day to about 80 mg/day of Compound I. In some embodiments, a method of inhibiting ITK in a human subject comprising administering an oral dose of about 10 mg/day to about 80 mg/day of Compound I.

The present invention also relates to a method of inhibiting at least one ITK function comprising the step of contacting ITK with Compound I as described herein. The cell phenotype, cell proliferation, activity of ITK, change in biochemical output produced by active ITK, expression of ITK, or binding of ITK with a natural binding partner may be monitored. Such methods may be modes of treatment of disease, biological assays, cellular assays, biochemical assays, or the like.

Compound I of the present invention may be useful as JAK3 kinase inhibitors as well as inhibitors of ITK. This dual use of a single compound inhibiting either ITK or JAK3 pathways individually or both pathways simultaneously may lead to a beneficial effect. As a result, Compound I of the present invention may find utility in treating a broad range of diseases or conditions mediated by ITK activity, JAK3 activity, or both. Accordingly, some embodiments herein are directed to treatment or prophylaxis of a disease or condition in which JAK, particularly JAK3, plays an active role using Compound I as disclosed herein. In some embodiments, a method of treating a JAK3 mediated disease or disorder in a human subject comprises administering to the human subject a compound of embodiments herein. Some embodiments provide methods for treating a JAK3-mediated disorder in a human subject in need of such treatment comprising administering to the human subject a therapeutically effective amount of a compound or composition according to the present disclosure. Also provided is the use of Compound I as disclosed herein for use in the manufacture of a medicament for the treatment of a disease or condition ameliorated by the inhibition of JAK3, ITK, or a combination thereof. Embodiments discussed herein which are directed to methods of treatment of ITK-mediated conditions or diseases may also apply to methods of treatment for JAK3 mediated conditions or diseases.

Also provided herein is a method of treating an ITK-mediated or JAK3 mediated disease comprising administering an oral dose of about 10 mg/day to about 80 mg/day of Compound I, a derivative thereof, or a combination thereof. In certain embodiments, oral dose of about 10 mg/day to about 80 mg/day of Compound I, a derivative thereof, or a combination thereof, may be in the form of a pharmaceutical composition. In embodiments, the pharmaceutical composition may include a pharmaceutically acceptable excipient.

In some embodiments, the present invention provides a method for treating an ITK mediated or JAK3 mediated disease or disorder in a human subject in need thereof, wherein said method comprises orally administering to the human subject an oral dose selected from about 10 mg/day to about 80 mg/day of Compound I, wherein the ITK-mediated or JAK3 mediated disease or disorder is an autoimmune disorders, immune mediated disorders, chronic inflammatory disorders, acute inflammatory disorders, and/or auto-inflammatory disorders and such disease or disorder is treated. In some embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID. Such ITK-mediated diseases or disorders include, but are not limited to, those described herein.

In some embodiments, diseases or disorders associated with an ITK kinase or a JAK3 kinase that are treated by compounds of the present invention include autoimmune disorders, immune mediated disorders, chronic inflammatory disorders, acute inflammatory disorders, and/or auto-inflammatory disorders.

In some embodiments, the ITK mediated or JAK3 mediated disease or disorder is arthritis and arthropathies. Arthritis and arthropathies include but are not limited to: idiopathic arthritis; osteoarthritis; rheumatoid arthritis; juvenile idiopathic arthritis; systemic-onset juvenile idiopathic arthritis; adult-onset Still's disease; seronegative spondyloarthropathies; spondyloarthropathy; ankylosing spondylitis; spondylitis; spondyloarthritis; enthesitis; enthesopathy; psoriatic arthritis; reactive arthritis; inflammatory arthritis; inflammatory bowel disease-associated arthritis; cystic fibrosis associated arthritis; gout; Reiter's syndrome; and Lyme-associated arthritis.

In some embodiments, the ITK mediated or JAK3 mediated disease or disorder is myositis and inflammatory myopathies. Myositis and inflammatory myopathies include but are not limited to: autoimmune myositis; dermatomyositis; polymyositis; and juvenile dermatomyositis.

In some embodiments, the ITK mediated or JAK3 mediated disease or disorder is scleroderma. Scleroderma include but is not limited to: systemic sclerosis/scleroderma; progressive systemic sclerosis; CREST syndrome (calcinosis; raynauds; esophageal dysfunction; sclerodactyly; telangiectasias); systemic sclerosis sine scleroderma; localized scleroderma; morphea; linear scleroderma; en coup de sabre scleroderma; and juvenile scleroderma.

In some embodiments, the ITK mediated or JAK3 mediated disease or disorder is lupus. Lupus includes but is not limited to: systemic lupus erythematosus; pediatric systemic lupus erythematosus; cutaneous lupus; subacute cutaneous lupus; chronic cutaneous lupus: discoid lupus; lupus erythematosus; chilblain lupus erythematosus; and lupus-associated specific organ manifestations (e.g., lupus nephritis; lupus arthritis; CNS lupus; etc.).

In some embodiments, the ITK mediated or JAK3 mediated disease or disorder is an autoinflammatory condition. Autoinflammatory conditions include but are not limited to: VEXAS syndrome; Muckle-Wells syndrome; familial cold auto-inflammatory syndrome: neonatal-onset multisystem inflammatory disease (NOMID); TNF receptor associated periodic syndrome (TRAPS); familial Mediterranean fever; cryopyrin-associated periodic syndrome (CAPS); auto-inflammatory disorders; and hyper IgD syndrome/mevalonate kinase deficiency.

In some embodiments, the ITK mediated or JAK3 mediated disease or disorder is vasculitis. Vasculitis includes but is not limited to: large vessel vasculitis; medium vessel vasculitis; small vessel vasculitis; ANCA+vasculitis; ANCA negative vasculitis; Takayasu's arteritis; Wegener's granulomatosis; giant-cell arteritis; polyarteritis nodosa; and Kawasaki Disease.

In some embodiments, the ITK mediated or JAK3 mediated disease or disorder is a gastrointestinal and/or a liver disorder. Gastrointestinal and liver disorders include but are not limited to: enteritis; colitis; enterocolitis; gastritis; esophagitis; inflammatory bowel disease; Crohn's disease; ulcerative colitis; irritable bowel syndrome; spastic colon; acute and chronic pancreatitis; celiac disease; periodontitis; gingivitis; eosinophilic gastritis; gastric and duodenal ulcers; peritonitis; autoimmune atrophic gastritis of pernicious anemia; alimentary track or gastrointestinal fibrosis; primary biliary cholangitis; primary sclerosing cholangitis; hepatic or biliary fibrosis; liver fibrosis (secondary to, for example, nonalcoholic steatohepatitis; hepatitis C; or hepatocellular carcinoma); cirrhosis (secondary to, for example, primary biliary cholangitis or cirrhosis due to fatty liver disease (for example, alcoholic and nonalcoholic steatosis); autoimmune and inflammatory hepatitis/liver disease; cholestasis; cholestatic liver injury/disease; and intrahepatic cholestasis of pregnancy.

In some embodiments, the ITK mediated or JAK3 mediated disease or disorder is a pulmonary-respiratory disorder. Pulmonary-respiratory disorders include but are not limited to: pulmonary inflammation; sinusitis; rhinitis; pneumonia; bronchitis; pulmonary fibrosis; idiopathic pulmonary fibrosis; asthma; bronchial asthma; allergic asthma; eosinophilic asthma; bronchial asthma; Churg-Strauss syndrome; bronchiolitis; bronchiolitis obliterans; chronic obstructive pulmonary disease (COPD); interstitial lung disease); acute lung injury; adult respiratory distress syndrome; drug-induced lung injury; and autoimmune interstitial lung disease.

In some embodiments, the ITK mediated or JAK3 mediated disease or disorder is an endocrine and/or a metabolic disorder. Endocrine and metabolic disorders include but are not limited to: diabetes mellitus; diabetes; Type I diabetes; autoimmune thyroid disorders; Hashimoto's thyroiditis; Graves' disease; Addison's disease; autoimmune ovarian failure; obesity; steroid-resistance; glucose intolerance; metabolic syndrome; metabolic bone disorders; thyroid illness; metabolic disorders; hyperparathyroidism; and menopause.

In some embodiments, the ITK mediated or JAK3 mediated disease or disorder is a hematologic disorder. Hematologic disorders include but are not limited to: autoimmune hemolytic syndromes; autoimmune hemolytic anemia; autoimmune thrombocytopenia: secondary hematologic manifestations of autoimmune diseases (e.g., anemias); polycythemia vera; essential thrombocythemia; iron deficiency anemia; myeloid metaplasia with myelofibrosis (MMM); primary myelofibrosis (PMF); and immune-mediated bone marrow failure disorders.

In some embodiments, the ITK mediated or JAK3 mediated disease or disorder is a neurologic disorder and/or a psychiatric disorder. Neurologic disorders and psychiatric disorders include but are not limited to: autoimmune encephalomyelitis; PANDA syndrome; neuromuscular disorders; neurodegenerative disorders; multiple sclerosis; Parkinson's disease: Huntington's disease; amyotrophic lateral sclerosis (ALS); familial ALS; Alzheimer's disease; myasthenia gravis Lambert-Eaton myasthenic syndrome (LEMS); Guillain-Barre syndrome; meningitis; encephalitis; traumatic brain injury; stroke; spinal cord injury; neuronal ischemia; peripheral neuropathy; CNS scarring; nerve irritation; nervous system damage; dysregulation of neuronal processes and sensory perception; peripheral neuropathy; psychogenic itch; tactile hallucinations; paresthetica cerebrovascular disorders; delusional parasitosis; notalgia obsessive-compulsive disorders; transverse myelitis; optic neuritic; and neuromyelitis optic.

In some embodiments, the ITK mediated or JAK3 mediated disease or disorder is a renal disease. Renal diseases include but are not limited to: nephropathies; IgA nephropathy: immunologically mediated glomerulonephropathy; autoimmune nephropathy; membranous glomerulopathy; chronic progressive nephropathies; diabetic nephropathy; renal fibrosis; ischemic/reperfusion injury associated; HIV associated nephropathy; ureteral obstructive nephropathy; glomerulosclerosis; nephrotic syndrome; proteinuria; polycystic kidney disease; autosomal dominant polycystic kidney disease; diabetic kidney disease; nephritis: glomerulonephritis; diffuse proliferative glomerulonephritis; focal segmental glomerulonephritis; membranous glomerulonephritis; lupus glomerulonephritis; immune complex glomerulonephritis; fibrillary glomerulonephritis; chronic kidney disease; diabetic nephropathy; hypertension induced nephropathy; an immunologically mediated nephropathy: uremia; and glomerulosclerosis disorders.

In some embodiments, the ITK mediated or JAK3 mediated disease or disorder is an ocular disorder. Ocular disorders include but are not limited to: dry eye; anterior uveitis: posterior uveitis; acute uveitis; chronic uveitis; keratoconjunctivitis sicca; scleritis; episcleritis: keratitis; keratopathy; chorditis; retinal vasculitis; optic neuritis; retinopathy; diabetic retinopathy; immune mediated retinopathy; macular degeneration; wet macular degeneration; dry (age related) macular degeneration; ocular malignancies; ocular inflammation; ophthalmic scarring; and sympathetic ophthalmia.

In some embodiments, the ITK mediated or JAK3 mediated disease or disorder is an allergy and/or an allergic reaction. Allergy and allergic reactions include but are not limited to: atopy; hypersensitivity reactions such as Type I hypersensitivity reactions including anaphylaxis; Type II hypersensitivity reactions including Goodpasture's Disease and autoimmune hemolytic anemia; Type III hypersensitivity reaction diseases including the Arthus reaction and serum sickness; and Type IV hypersensitivity reactions including contact dermatitis; and allograft rejection.

In some embodiments, the ITK mediated or JAK3 mediated disease or disorder is dermatologic condition. Dermatologic conditions include but are not limited to: a skin disorder; atopy; atopic dermatitis; eczema; contact dermatitis; allergic contact sensitization; allergic dermatitis; acne; acne vulgaris; comedonal acne; inflammatory acne; nodulo-cystic acne; scarring acne; acne keloidalis nuchae; hidradenitis suppurativa; neutrophilic dermatoses; pyoderma gangrenosum; acute febrile neutrophilic dermatosis (Sweet's syndrome); erythema elevatinum diutinum (EED); neutrophilic eccrine hidradenitis; histiocytoid neutrophilic dermatosis; bowel-bypass syndrome dermatosis; palisading neutrophilic granulomatous dermatitis; neutrophilic urticarial dermatosis; chronic atypical neutrophilic dermatosis with lipodystrophy and elevated temperature syndrome (CANDLE Syndrome); a hair loss disorder; alopecia; nonscarring alopecia; alopecia areata (AA); patchy AA; alopecia totalis (AT); alopecia universalis (AU); androgenetic alopecia (AGA); male and female pattern AGA; ophiasis pattern alopecia areata; sisaihpo pattern alopecia areata; telogen effluvium; tinea capitis; hypotrichosis; hereditary hypotrichosis simplex; scarring alopecia; lichen planopilaris: central centrifugal cicatricial alopecia; frontal fibrosing alopecia; eyebrow alopecia; intranasal hair alopecia; vitiligo; segmental vitiligo; unisegmental; bisegmental or multisegmental vitiligo; non-segmental vitiligo including acral; facial; or acrofacial vitiligo; centrofacial vitiligo; mucosal vitiligo; confetti vitiligo; trichrome vitiligo; marginal inflammatory vitiligo: quadrichrome vitiligo; blue vitiligo; Koebner phenomenon; vulgaris vitiligo; generalized vitiligo; universal vitiligo; mixed vitiligo (nonsegmental associated with segmental vitiligo); focal vitiligo; solitary mucosal vitiligo or vitiligo with or without leukotricia (involvement of body hair); bullous diseases; immunobullous diseases; bullous pemphigoid; cicatricial pemphigoid; pemphigus vulgaris; linear IgA disease; dermatologic drug reactions; idiopathic chronic itch; pruritus (itch) conditions; atopic pruritus; atopic dermatitis-associated itch: xerotic pruritus; pruritus associated with psoriasis (“psoriatic itch”); acute pruritus; chronic pruritus; idiopathic pruritus; brachioradial pruritus; neurogenic itch; neuropathic itch; chronic idiopathic pruritus; hepatobiliary-associated itch; biliary itch; renal associated itch; uremic itch; lichen simplex chronicus associated pruritus; prurigo nodularis; Type II diabetes associated itch; hypophysitis associated itch; idiopathic thrombocytopenic purpura-associated itch; pruritic urticarial papules and plaques of pregnancy (PUPP); swimmer's itch; lymphoma-associated itch; leukemia-associated itch; eruption of HIV; punctate palmoplantar keratoderma; psoriasis; psoriasis vulgaris; skin sensitization; skin irritation; skin rash; inflammatory dermatoses; drug cruptions; dermatosis of pregnancy; cosinophilic folliculitis: foreign objects or devices on skin; fungal infection of the skin; gestational pemphigoid; dandruff; photodermatitis; scar growth; seborieic dermatitis; stasis dermatitis; sunburn; hives: urticaria; urticarial itch; papular urticaria; chronic idiopathic urticaria; chronic urticaria; wound healing; wound or scab healing; surgical scarring; lichen planus; lichen sclerosis; insect bites; insect stings; notalgia paresthetica; and xerosis.

In some embodiments, the ITK mediated or JAK3 mediated disease or disorder is an infection and/or an infestation. Infections and infestations include but are not limited to: acute and chronic infection; sepsis syndromes; sepsis; septic shock; endotoxic shock; exotoxin-induced toxic shock; gram negative sepsis; gram positive sepsis; fungal sepsis; toxic shock syndrome; bacterial infection; body louse; pruritic popular pubic lice; head lice; herpes: cutaneous larva migrans; scabies; varicella; viral infection; parasitic infection; pediculosis: insect infestation; infectious arthritis; and Lyme disease.

In some embodiments, the ITK mediated or JAK3 mediated disease or disorder is an oncologic condition. Oncologic conditions include but are not limited to: a cancer; neoplasias; internal cancer; malignancy; a myeloproliferative disorder; a hematopoietic neoplasm; a myeloid neoplasm; a lymphoid neoplasm; non-small cell lung carcinoma; small cell lung carcinoma; skin cancer; a myeloproliferative disorder; myelodysplastic syndrome; leukemias (e.g., acute and chronic leukemias; acute lymphocytic leukemia; acute and chronic myelogenous leukemia; chronic lymphocytic leukemia; acute lymphoblastic leukemia; or promyelocytic leukemia; chronic myelomonocytic leukemia); lymphomas (e.g., B-cell lymphoma; T-cell lymphoma; mantle cell lymphoma; hairy cell lymphoma; Burkitt's lymphoma; mast cell tumors; Hodgkin's disease or non-Hodgkin's disease; cutaneous lymphomas including mycosis fungoides and cutaneous T-cell lymphoma; myeloid malignancies; myelodysplastic syndrome; fibrosarcoma; rhabdomyosarcoma; brain and nervous system tumors (e.g., glioma or glioblastoma multiforme; astrocytoma; neuroblastoma; glioma; schwannomas; retinoblastoma) melanoma; seminoma; teratoma; teratocarcinoma; osteosarcoma; xeroderma pigmentosum; keratoacanthoma; thyroid cancer; thyroid follicular cancer; Kapo′i's sarcoma; prostate cancer; renal cancer; hepatic/hepatobiliary cancer; breast cancer; pancreatic cancer; multiple myeloma; cancer of the bone; mouth/pharynx; esophagus: larynx; stomach; intestine; colon; rectum; liver; nerve; head and neck; throat, ovary; uterus; testis; bladder; kidney; gall bladder; cervix; fibrotic cancers; fibroids; fibroma; fibroadenomas; fibrosarcomas; metastatic bone disorders and lesions.

In some embodiments, the ITK mediated or JAK3 mediated disease or disorder is a transplant related treatment or preventative measure. Transplant related treatments or preventative measures include but are not limited to: prevention or treatment of organ transplant rejection; prevention or treatment of bone marrow transplant rejection; graft vs. host reaction; graft vs. host disease; chronic graft versus host disease; acute graft vs host disease: allograft rejections; acute allograft rejection; and chronic allograft rejection.

In some embodiments, the ITK mediated or JAK3 mediated disease or disorder is a cardiovascular disorders Cardiovascular disorders include but are not limited to: atherosclerosis; restenosis; restenosis of an atherosclerotic coronary artery; acute coronary syndrome; myocardial infarction; cardiac-allograft vasculopathy; cardiac fibrosis: endomyocardial fibrosis; atrial fibrosis; recurrent pericarditis; chronic pericarditis; myocarditis; carditis; myocarditis; and angiogenesis disorders.

In some embodiments, the ITK mediated or JAK3 mediated disease or disorder is pain disorder (e.g., acute pain; chronic pain; neuropathic pain; or fibromyalgia).

In some embodiments, the ITK mediated or JAK3 mediated disease or disorder is a fibrotic disorder. Fibrotic disorders include but are not limited to: disorders of fibrosis and scarring (e.g., hepatic fibrosis; pulmonary fibrosis; idiopathic pulmonary fibrosis; low grade scarring such as; scleroderma; increased fibrosis; keloids; post-surgical scars); myelofibrosis; primary myelofibrosis; idiopathic myelofibrosis (IMF); radiation induced fibrosis (e.g., head and neck; gastrointestinal or pulmonary); fibrosclerosis; mediastinal fibrosis; retroperitoneal fibrosis; progressive massive fibrosis; and nephrogenic systemic fibrosis.

In some embodiments, the ITK mediated or JAK3 mediated disease or disorder is inflammation; an inflammatory condition; chronic inflammatory disorders; acute inflammatory disorders; connective tissue diseases; an autoimmune conditions/disorders/responses: autoimmune connective tissue disease; Sjogren's syndrome; mixed connective tissue disease; polymyalgia rheumatica; sarcoidosis; autoimmune orchitis; Goodpasture's disease; Behcet's disease; metal-induced autoimmunity; autoimmune deafness (including, for example, Meniere's disease); interstitial cystitis; polychondritis; relapsing polychondritis; autoimmune orchitis; silicone implant associated autoimmune disease; drug-induced autoimmunity; HIV-related autoimmune syndromes; broad activation of the immune responses; bone resorption diseases; SAVI (stimulator of interferon genes (STING) associated vasculopathy with onset in infancy); hyperoxia induced inflammations; reperfusion injury; post-surgical trauma; tissue injury; cystic fibrosis; Th17-associated inflammation; increased accumulation of exogenous opioids or synthetic opioids; jaundice; mastocytosis; Castleman's disease; systemic immune senescence; transplant arteriopathy; hypereosinophilic syndrome (HES); systemic mast cell disease (SMCD); and macrophage activation syndrome.

In some embodiments, the ITK-mediated or JAK3 mediated disease is selected from the group consisting of atopic dermatitis, palmoplantar pustulosis, fibrosing interstitial lung disease, peripheral T-cell lymphoma. Celiac disease, asthma, cryopyrin-associated periodic syndrome (CAPS), recurrent pericarditis, VEXAS syndrome, ulcerative colitis, acute severe ulcerative colitis, Crohn's disease, inflammatory bowel disease, rheumatoid arthritis, psoriatic arthritis, ankylosing spondylitis, acute inflammatory psoriasis, genital psoriasis, autoimmune hepatitis, systemic lupus erythematosus (SLE), organ transplant rejection, chronic graft versus host disease (cGvHD), primary sclerosing cholangitis, or a combination thereof.

Another embodiment of the present disclosure provides a method for treating an inflammatory condition in a human subject in need thereof comprising administering, to the human subject having an inflammatory condition, an oral dose of about 10 mg/day to about 80 mg/day of Compound I, wherein the inflammatory condition is selected from the group consisting of a chronic inflammatory condition, an acute inflammatory condition, an immuno-inflammatory condition, an autoimmune inflammatory condition, an inflammasomopathy, or a combination thereof and such condition is treated. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

In some embodiments, the inflammatory condition is selected from the group consisting of a chronic inflammatory condition, an acute inflammatory condition, an immuno-inflammatory condition, an autoimmune inflammatory condition, an inflammasomopathy, or a combination thereof.

In some embodiments, the inflammatory condition is selected from the group consisting of atopic dermatitis, palmoplantar pustulosis, fibrosing interstitial lung disease, peripheral T-cell lymphoma, Celiac disease, asthma, cryopyrin-associated periodic syndrome (CAPS), recurrent pericarditis, VEXAS syndrome, ulcerative colitis, acute severe ulcerative colitis, Crohn's disease, inflammatory bowel disease, rheumatoid arthritis, psoriatic arthritis, ankylosing spondylitis, acute inflammatory psoriasis, genital psoriasis, autoimmune hepatitis, systemic lupus erythematosus (SLE), organ transplant rejection, chronic graft versus host disease (cGvHD), primary sclerosing cholangitis, or a combination thereof.

In some embodiments the inflammatory condition is atopic dermatitis.

In some embodiments the inflammatory condition is palmoplantar pustulosis.

In some embodiments the inflammatory condition is fibrosing interstitial lung disease.

In some embodiments the inflammatory condition is peripheral T-cell lymphoma.

In some embodiments the inflammatory condition is Celiac disease.

In some embodiments the inflammatory condition is asthma.

In some embodiments the inflammatory condition is acute severe ulcerative colitis.

In some embodiments the inflammatory condition is Crohn's disease.

In some embodiments the inflammatory condition is psoriatic arthritis.

In some embodiments the inflammatory condition is ankylosing spondylitis.

In some embodiments the inflammatory condition is acute inflammatory psoriasis.

In some embodiments the inflammatory condition is genital psoriasis.

In some embodiments the inflammatory condition is autoimmune hepatitis.

In some embodiments the inflammatory condition is systemic lupus erythematosus (SLE).

In some embodiments the inflammatory condition is organ transplant rejection.

In some embodiments the inflammatory condition is chronic graft versus host disease (cGvHD).

In some embodiments the inflammatory condition is primary sclerosing cholangitis.

In some embodiments the inflammatory condition is cryopyrin-associated periodic syndrome (CAPS).

In some embodiments the inflammatory condition is recurrent pericarditis.

In some embodiments the inflammatory condition is VEXAS syndrome.

In some embodiments the inflammatory condition is ulcerative colitis.

In some embodiments the inflammatory condition is rheumatoid arthritis.

In some embodiments the inflammatory condition is inflammatory bowel disease.

In some embodiments the inflammation resulting from the inflammatory bowel disease is treated.

In some embodiments the inflammation resulting from inflammatory bowel disease is in the proximal portion of the bowel.

In some embodiments the inflammation resulting from inflammatory bowel disease is in the distal portion of the bowel.

In some embodiments the inflammation resulting from inflammatory bowel disease is in the ileum portion of the bowel.

In some embodiments the inflammation resulting from inflammatory bowel disease is in the entirety of the bowel.

In some embodiments, the human subject has a decreased incidence of anemia compared to the incidence of anemia if administered tofacitinib. In some embodiments, the human subject has less than a 4% chance of developing anemia. In some embodiments, the human subject has less than a 2% chance of developing anemia.

In some embodiments, the human subject has a decreased incidence of nephrotoxicity compared to the incidence of nephrotoxicity if administered ritlecitinib.

Another embodiment of the present disclosure provides a method for treating atopic dermatitis in a human subject in need thereof comprising administering to the human subject, an oral dose of about 10 mg/day to about 80 mg/day of Compound I. In some embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating palmoplantar pustulosis in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating fibrosing interstitial lung disease in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating peripheral T-cell lymphoma in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating Celiac disease in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating asthma in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating acute severe ulcerative colitis in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating Crohn's disease in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating psoriatic arthritis in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating ankylosing spondylitis in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating acute inflammatory psoriasis in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for genital psoriasis in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for autoimmune hepatitis in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for systemic lupus erythematosus (SLE) in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for organ transplant rejection in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for chronic graft versus host disease (cGvHD) in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for primary sclerosing cholangitis in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating cryopyrin-associated periodic syndrome (CAPS) in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating recurrent pericarditis in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating VEXAS syndrome in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating ulcerative colitis in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating rheumatoid arthritis in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating inflammatory bowel disease in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

Another embodiment of the present disclosure provides a method for treating inflammation resulting from the inflammatory bowel disease in a human subject in need thereof comprising administering to the human subject, an oral dose of about 10 mg/day to about 80 mg/day of Compound I. In embodiments, the oral dose is about 5 mg BID to about 40 mg BID. In some embodiments, the oral dose is about 30 mg/day. In some embodiments, the oral dose is about 5 mg BID. In some embodiments, the oral dose is about 15 mg BID. In some embodiments, the oral dose is about 25 mg BID. In some embodiments, the oral dose is about 40 mg BID. In some embodiments, the oral dose is about 10 mg TID.

As described supra, subjects suitable for treatment in accordance with the methods described herein include, humans and non-human vertebrates such as wild, domestic, and farm animals. In some embodiments, the subject described herein is an animal. In some embodiments, the subject is a mammal. In some embodiments, the subject is a human. In some embodiments, the subject is a non-human animal. In some embodiments, the subject is a non-human mammal.

### Dosing Regimen

Compound I, or an oral pharmaceutical composition comprising the same, is administered to a human subject in an oral dose of about 10 mg/day to about 80 mg/day of Compound I. Human subjects in need of such therapy are disclosed above. In some embodiments, the human subject is one having an inflammatory condition, e.g., a chronic inflammatory condition, an acute inflammatory condition, an immuno-inflammatory condition, an autoimmune condition, or an inflammasomopathy. In accordance with the methods disclosed herein, a therapeutically effective amount of about 10 mg/day to about 80 mg/day of Compound I. The dosage to be administered to a particular subject will depend on the characteristics of the subject being treated, e.g., the particular subject treated, age, weight, health, types of concurrent treatment, if any, and frequency of treatments, and can be easily determined by one of skill in the art (e.g., by the clinician).

In any embodiment, the therapeutically effective amount of 1 mg/day of Compound I is administered to a human subject having an inflammatory condition disclosed herein. In any embodiment, the therapeutically effective amount of 3 mg/day of Compound I is administered to a human subject having an inflammatory condition disclosed herein. In any embodiment, the therapeutically effective amount of 5 mg/day of Compound I is administered to a human subject having an inflammatory condition disclosed herein. In any embodiment, the therapeutically effective amount of 10 mg/day of Compound I is administered to a human subject having an inflammatory condition disclosed herein. In any embodiment, the therapeutically effective amount of 15 mg/day of Compound I is administered to a human subject having an inflammatory condition disclosed herein. In any embodiment, the therapeutically effective amount of 25 mg/day of Compound I is administered to a human subject having an inflammatory condition disclosed herein. In any embodiment, the therapeutically effective amount of 30 mg/day of Compound I is administered to a human subject having an inflammatory condition disclosed herein. In any embodiment, the therapeutically effective amount of 40 mg/day of Compound I is administered to a human subject having an inflammatory condition disclosed herein. In any embodiment, the therapeutically effective amount of 50 mg/day of Compound I is administered to a human subject having an inflammatory condition disclosed herein. In any embodiment, the therapeutically effective amount of 80 mg/day of Compound I is administered to a human subject having an inflammatory condition disclosed herein.

In any embodiment, the therapeutically effective amount of Compound I is about 1 mg/day to about 100 mg/day or any amount in between. In an embodiment, the therapeutically effective amount of Compound I is about 2 mg/day to about 100 mg/day. In an embodiment, the therapeutically effective amount of Compound I is about 3 mg/day to about 100 mg/day. In an embodiment, the therapeutically effective amount of Compound I is about 4 mg/day to about 100 mg/day. In an embodiment, the therapeutically effective amount of Compound I is about 5 mg/day to about 100 mg/day. In an embodiment, the therapeutically effective amount of Compound I is about 10 mg/day to about 100 mg/day. In an embodiment, the therapeutically effective amount of Compound I is about 15 mg/day to about 100 mg/day. In an embodiment, the therapeutically effective amount of Compound I is about 20 mg/day to about 100 mg/day. In an embodiment, the therapeutically effective amount of Compound I is about 25 mg/day to about 100 mg/day. In an embodiment, the therapeutically effective amount of Compound I is about 30 mg/day to about 100 mg/day. In an embodiment, the therapeutically effective amount of Compound I is about 35 mg/day to about 100 mg/day. In an embodiment, the therapeutically effective amount of Compound I is about 40 mg/day to about 100 mg/day. In an embodiment, the therapeutically effective amount of Compound I is about 45 mg/day to about 100 mg/day. In an embodiment, the therapeutically effective amount of Compound I is about 50 mg/day to about 100 mg/day. In an embodiment, the therapeutically effective amount of Compound I is about 1 mg/day to about 90 mg/day. In an embodiment, the therapeutically effective amount of Compound I is about 1 mg/day to about 80 mg/day. In an embodiment, the therapeutically effective amount of Compound I is about 1 mg/day to about 70 mg/day. In an embodiment, the therapeutically effective amount of Compound I is about 1 mg/day to about 60 mg/day. In an embodiment, the therapeutically effective amount of Compound I is about 1 mg/day to about 50 mg/day. In an embodiment, the therapeutically effective amount of Compound I is about 1 mg/day to about 40 mg/day. In an embodiment, the therapeutically effective amount of Compound I is about 1 mg/day to about 30 mg/day. In an embodiment, the therapeutically effective amount of Compound I is about 1 mg/day to about 20 mg/day. In an embodiment, the therapeutically effective amount of Compound I is about 1 mg/day to about 10 mg/day. In an embodiment the therapeutically effective amount of Compound I is about 10 mg/day to about 80 mg/day. In an embodiment, the therapeutically effective amount of Compound I comprises about 0.1 mg/day. In an embodiment, the therapeutically effective amount of Compound I comprises about 0.5 mg/day. In an embodiment, the therapeutically effective amount of Compound I comprises about 1 mg/day. In an embodiment, the therapeutically effective amount of Compound I comprises about 2 mg/day. In an embodiment, the therapeutically effective amount of Compound I comprises about 3 mg/day. In an embodiment, the therapeutically effective amount of Compound I comprises about 4 mg/day. In an embodiment, the therapeutically effective amount of Compound I comprises about 5 mg/day. In an embodiment, the therapeutically effective amount of Compound I comprises about 6 mg/day. In an embodiment, the therapeutically effective amount of Compound I comprises about 7 mg/day. In an embodiment, the therapeutically effective amount of Compound I comprises about 8 mg/day. In an embodiment, the therapeutically effective amount of Compound I comprises about 9 mg/day. In an embodiment, the therapeutically effective amount of Compound I comprises about 10 mg/day. In an embodiment, the therapeutically effective amount of Compound I comprises about 11 mg/day. In an embodiment, the therapeutically effective amount of Compound I comprises about 12 mg/day. In an embodiment, the therapeutically effective amount of Compound I comprises about 13 mg/day. In an embodiment, the therapeutically effective amount of Compound I comprises about 14 mg/day. In an embodiment, the therapeutically effective amount of Compound I comprises about 15 mg/day. In an embodiment, the therapeutically effective amount of Compound I comprises about 16 mg/day. In an embodiment, the therapeutically effective amount of Compound I comprises about 17 mg/day. In an embodiment, the therapeutically effective amount of Compound I comprises about 18 mg/day. In an embodiment, the therapeutically effective amount of Compound I comprises about 19 mg/day. In an embodiment, the therapeutically effective amount of Compound I comprises about 20 mg/day. In an embodiment, the therapeutically effective amount of Compound I comprises about 21 mg/day. In an embodiment, the therapeutically effective amount of Compound I comprises about 22 mg/day. In an embodiment, the therapeutically effective amount of Compound I comprises about 23 mg/day. In an embodiment, the therapeutically effective amount of Compound I comprises about 24 mg/day. In an embodiment, the therapeutically effective amount of Compound I comprises about mg/day. In an embodiment, the therapeutically effective amount of Compound I comprises about 26 mg/day. In an embodiment, the therapeutically effective amount of Compound I comprises about 27 mg/day. In an embodiment, the therapeutically effective amount of Compound I comprises about 28 mg/day. In an embodiment, the therapeutically effective amount of Compound I comprises about 29 mg/day. In an embodiment, the therapeutically effective amount of Compound I comprises about 30 mg/day. In an embodiment, the therapeutically effective amount of Compound I comprises about 31 mg/day. In an embodiment, the therapeutically effective amount of Compound I comprises about 32 mg/day. In an embodiment, the therapeutically effective amount of Compound I comprises about 33 mg/day. In an embodiment, the therapeutically effective amount of Compound I comprises about 34 mg/day. In an embodiment, the therapeutically effective amount of Compound I comprises about 35 mg/day. In an embodiment, the therapeutically effective amount of Compound I comprises about 36 mg/day. In an embodiment, the therapeutically effective amount of Compound I comprises about 37 mg/day. In an embodiment, the therapeutically effective amount of Compound I comprises about 38 mg/day. In an embodiment, the therapeutically effective amount of Compound I comprises about 39 mg/day. In an embodiment, the therapeutically effective amount of Compound I comprises about 40 mg/day. In an embodiment, the therapeutically effective amount of Compound I comprises about 41 mg/day. In an embodiment, the therapeutically effective amount of Compound I comprises about 42 mg/day. In an embodiment, the therapeutically effective amount of Compound I comprises about 43 mg/day. In an embodiment, the therapeutically effective amount of Compound I comprises about 44 mg/day. In an embodiment, the therapeutically effective amount of Compound I comprises about 45 mg/day. In an embodiment, the therapeutically effective amount of Compound I comprises about 46 mg/day. In an embodiment, the therapeutically effective amount of Compound I comprises about 47 mg/day. In an embodiment, the therapeutically effective amount of Compound I comprises about 48 mg/day. In an embodiment, the therapeutically effective amount of Compound I comprises about 49 mg/day. In an embodiment, the therapeutically effective amount of Compound I comprises about 50 mg/day. In an embodiment, the therapeutically effective amount of Compound I comprises about 51 mg/day. In an embodiment, the therapeutically effective amount of Compound I comprises about 52 mg/day. In an embodiment, the therapeutically effective amount of Compound I comprises about 53 mg/day. In an embodiment, the therapeutically effective amount of Compound I comprises about 54 mg/day. In an embodiment, the therapeutically effective amount of Compound I comprises about 55 mg/day. In an embodiment, the therapeutically effective amount of Compound I comprises about 56 mg/day. In an embodiment, the therapeutically effective amount of Compound I comprises about 57 mg/day. In an embodiment, the therapeutically effective amount of Compound I comprises about 58 mg/day. In an embodiment, the therapeutically effective amount of Compound I comprises about 59 mg/day. In an embodiment, the therapeutically effective amount of Compound I comprises about 60 mg/day. In an embodiment, the therapeutically effective amount of Compound I comprises about 61 mg/day. In an embodiment, the therapeutically effective amount of Compound I comprises about 62 mg/day. In an embodiment, the therapeutically effective amount of Compound I comprises about 63 mg/day. In an embodiment, the therapeutically effective amount of Compound I comprises about mg/day. In an embodiment, the therapeutically effective amount of Compound I comprises about mg/day. In an embodiment, the therapeutically effective amount of Compound I comprises about 66 mg/day. In an embodiment, the therapeutically effective amount of Compound I comprises about 67 mg/day. In an embodiment, the therapeutically effective amount of Compound I comprises about 68 mg/day. In an embodiment, the therapeutically effective amount of Compound I comprises about 69 mg/day. In an embodiment, the therapeutically effective amount of Compound I comprises about 70 mg/day. In an embodiment, the therapeutically effective amount of Compound I comprises about 71 mg/day. In an embodiment, the therapeutically effective amount of Compound I comprises about 72 mg/day. In an embodiment, the therapeutically effective amount of Compound I comprises about 73 mg/day. In an embodiment, the therapeutically effective amount of Compound I comprises about 74 mg/day. In an embodiment, the therapeutically effective amount of Compound I comprises about 75 mg/day. In an embodiment, the therapeutically effective amount of Compound I comprises about 76 mg/day. In an embodiment, the therapeutically effective amount of Compound I comprises about 77 mg/day. In an embodiment, the therapeutically effective amount of Compound I comprises about 78 mg/day. In an embodiment, the therapeutically effective amount of Compound I comprises about 79 mg/day. In an embodiment, the therapeutically effective amount of Compound I comprises about 80 mg/day. In an embodiment, the therapeutically effective amount of Compound I comprises about 81 mg/day. In an embodiment, the therapeutically effective amount of Compound I comprises about 82 mg/day. In an embodiment, the therapeutically effective amount of Compound I comprises about 83 mg/day. In an embodiment, the therapeutically effective amount of Compound I comprises about 84 mg/day. In an embodiment, the therapeutically effective amount of Compound I comprises about 85 mg/day. In an embodiment, the therapeutically effective amount of Compound I comprises about 86 mg/day. In an embodiment, the therapeutically effective amount of Compound I comprises about 87 mg/day. In an embodiment, the therapeutically effective amount of Compound I comprises about 88 mg/day. In an embodiment, the therapeutically effective amount of Compound I comprises about 89 mg/day, about 90 mg/day. In an embodiment, the therapeutically effective amount of Compound I comprises about 91 mg/day. In an embodiment, the therapeutically effective amount of Compound I comprises about 92 mg/day. In an embodiment, the therapeutically effective amount of Compound I comprises about 93 mg/day. In an embodiment, the therapeutically effective amount of Compound I comprises about 94 mg/day. In an embodiment, the therapeutically effective amount of Compound I comprises about 95 mg/day, about 96 mg/day, about 97 mg/day. In an embodiment, the therapeutically effective amount of Compound I comprises about 98 mg/day, about 99 mg/day. In an embodiment, the therapeutically effective amount of Compound I comprises about 100 mg/day.

In any embodiment, the therapeutically effective amount of 5 mg BID of Compound I is administered to a human subject having an inflammatory condition disclosed herein. In any embodiment, the therapeutically effective amount of 10 mg BID of Compound I is administered to a human subject having an inflammatory condition disclosed herein. In any embodiment, the therapeutically effective amount of 15 mg BID of Compound I is administered to a human subject having an inflammatory condition disclosed herein. In any embodiment, the therapeutically effective amount of 25 mg BID of Compound I is administered to a human subject having an inflammatory condition disclosed herein. In any embodiment, the therapeutically effective amount of 40 mg BID of Compound I is administered to a human subject having an inflammatory condition disclosed herein.

In any embodiment, the therapeutically effective amount of Compound I is about 1 mg BID to about 100 mg BID or any amount in between. In an embodiment, the therapeutically effective amount of Compound I is about 2 mg BID to about 50 mg BID. In an embodiment, the therapeutically effective amount of Compound I is about 3 mg BID to about 50 mg BID. In an embodiment, the therapeutically effective amount of Compound I is about 4 mg BID to about 50 mg BID. In an embodiment, the therapeutically effective amount of Compound I is about 5 mg BID to about 50 mg BID. In an embodiment, the therapeutically effective amount of Compound I is about 10 mg BID to about 50 mg BID. In an embodiment, the therapeutically effective amount of Compound I is about 15 mg BID to about 50 mg BID. In an embodiment, the therapeutically effective amount of Compound I is about 20 mg BID to about 50 mg BID. In an embodiment, the therapeutically effective amount of Compound I is about 25 mg BID to about 50 mg BID. In an embodiment, the therapeutically effective amount of Compound I is about 30 mg BID to about 50 mg BID. In an embodiment, the therapeutically effective amount of Compound I is about 35 mg BID to about 100 mg BID. In an embodiment, the therapeutically effective amount of Compound I is about 40 mg BID to about 50 mg BID. In an embodiment, the therapeutically effective amount of Compound I is about 45 mg BID to about 50 mg BID. In an embodiment, the therapeutically effective amount of Compound I is about 1 mg BID to about 45 mg BID. In an embodiment, the therapeutically effective amount of Compound I is about 1 mg BID to about 40 mg BID. In an embodiment, the therapeutically effective amount of Compound I is about 1 mg BID to about 35 mg BID. In an embodiment, the therapeutically effective amount of Compound I is about 1 mg BID to about 30 mg BID. In an embodiment, the therapeutically effective amount of Compound I is about 1 mg BID to about 25 mg BID. In an embodiment, the therapeutically effective amount of Compound I is about 1 mg BID to about 20 mg BID. In an embodiment, the therapeutically effective amount of Compound I is about 1 mg BID to about 15 mg BID. In an embodiment, the therapeutically effective amount of Compound I is about 1 mg BID to about 10 mg BID. In an embodiment, the therapeutically effective amount of Compound I is about 1 mg BID to about 5 mg BID. In an embodiment the therapeutically effective amount of Compound I is about 1 mg BID to about 50 mg BID. In an embodiment the therapeutically effective amount of Compound I is about 5 mg BID to about 40 mg BID. In an embodiment, the therapeutically effective amount of Compound I comprises about 1 mg BID. In an embodiment, the therapeutically effective amount of Compound I comprises about 2 mg BID. In an embodiment, the therapeutically effective amount of Compound I comprises about 3 mg BID. In an embodiment, the therapeutically effective amount of Compound I comprises about 4 mg BID. In an embodiment, the therapeutically effective amount of Compound I comprises about 5 mg BID. In an embodiment, the therapeutically effective amount of Compound I comprises about 6 mg BID. In an embodiment, the therapeutically effective amount of Compound I comprises about 7 mg BID. In an embodiment, the therapeutically effective amount of Compound I comprises about 8 mg BID. In an embodiment, the therapeutically effective amount of Compound I comprises about 9 mg BID. In an embodiment, the therapeutically effective amount of Compound I comprises about 10 mg BID. In an embodiment, the therapeutically effective amount of Compound I comprises about 11 mg BID. In an embodiment, the therapeutically effective amount of Compound I comprises about 12 mg BID. In an embodiment, the therapeutically effective amount of Compound I comprises about 13 mg BID. In an embodiment, the therapeutically effective amount of Compound I comprises about 14 mg BID. In an embodiment, the therapeutically effective amount of Compound I comprises about 15 mg BID. In an embodiment, the therapeutically effective amount of Compound I comprises about mg 16 BID. In an embodiment, the therapeutically effective amount of Compound I comprises about 17 mg BID. In an embodiment, the therapeutically effective amount of Compound I comprises about 18 mg BID. In an embodiment, the therapeutically effective amount of Compound I comprises about 19 mg BID. In an embodiment, the therapeutically effective amount of Compound I comprises about 20 mg BID. In an embodiment, the therapeutically effective amount of Compound I comprises about 21 mg BID. In an embodiment, the therapeutically effective amount of Compound I comprises about 22 mg BID. In an embodiment, the therapeutically effective amount of Compound I comprises about 23 mg BID. In an embodiment, the therapeutically effective amount of Compound I comprises about 24 mg BID. In an embodiment, the therapeutically effective amount of Compound I comprises about 25 mg BID. In an embodiment, the therapeutically effective amount of Compound I comprises about 26 mg BID. In an embodiment, the therapeutically effective amount of Compound I comprises about 27 mg BID. In an embodiment, the therapeutically effective amount of Compound I comprises about 28 mg BID. In an embodiment, the therapeutically effective amount of Compound I comprises about 29 mg BID. In an embodiment, the therapeutically effective amount of Compound I comprises about 30 mg BID. In an embodiment, the therapeutically effective amount of Compound I comprises about 31 mg BID. In an embodiment, the therapeutically effective amount of Compound I comprises about 32 mg BID. In an embodiment, the therapeutically effective amount of Compound I comprises about 33 mg BID. In an embodiment, the therapeutically effective amount of Compound I comprises about 34 mg BID. In an embodiment, the therapeutically effective amount of Compound I comprises about 35 mg BID. In an embodiment, the therapeutically effective amount of Compound I comprises about 36 mg BID. In an embodiment, the therapeutically effective amount of Compound I comprises about 37 mg BID. In an embodiment, the therapeutically effective amount of Compound I comprises about 38 mg BID. In an embodiment, the therapeutically effective amount of Compound I comprises about 39 mg BID. In an embodiment, the therapeutically effective amount of Compound I comprises about 40 mg BID. In an embodiment, the therapeutically effective amount of Compound I comprises about 41 mg BID. In an embodiment, the therapeutically effective amount of Compound I comprises about 42 mg BID. In an embodiment, the therapeutically effective amount of Compound I comprises about 43 mg BID. In an embodiment, the therapeutically effective amount of Compound I comprises about 44 mg BID. In an embodiment, the therapeutically effective amount of Compound I comprises about 45 mg BID. In an embodiment, the therapeutically effective amount of Compound I comprises about 46 mg BID. In an embodiment, the therapeutically effective amount of Compound I comprises about 47 mg BID. In an embodiment, the therapeutically effective amount of Compound I comprises about 48 mg BID. In an embodiment, the therapeutically effective amount of Compound I comprises about 49 mg BID. In an embodiment, the therapeutically effective amount of Compound I comprises about 50 mg BID. In an embodiment, the therapeutically effective amount of Compound I comprises about 51 mg BID. In an embodiment, the therapeutically effective amount of Compound I comprises about 52 mg BID. In an embodiment, the therapeutically effective amount of Compound I comprises about 53 mg BID. In an embodiment, the therapeutically effective amount of Compound I comprises about 54 mg BID. In an embodiment, the therapeutically effective amount of Compound I comprises about 55 mg BID. In an embodiment, the therapeutically effective amount of Compound I comprises about 56 mg BID. In an embodiment, the therapeutically effective amount of Compound I comprises about 57 mg BID. In an embodiment, the therapeutically effective amount of Compound I comprises about 58 mg BID. In an embodiment, the therapeutically effective amount of Compound I comprises about 59 mg BID. In an embodiment, the therapeutically effective amount of Compound I comprises about 60 mg BID. In an embodiment, the therapeutically effective amount of Compound I comprises about 61 mg BID. In an embodiment, the therapeutically effective amount of Compound I comprises about 62 mg BID. In an embodiment, the therapeutically effective amount of Compound I comprises about 63 mg BID. In an embodiment, the therapeutically effective amount of Compound I comprises about 64 mg BID. In an embodiment, the therapeutically effective amount of Compound I comprises about 65 mg BID. In an embodiment, the therapeutically effective amount of Compound I comprises about 66 mg BID. In an embodiment, the therapeutically effective amount of Compound I comprises about 67 mg BID. In an embodiment, the therapeutically effective amount of Compound I comprises about 68 mg BID. In an embodiment, the therapeutically effective amount of Compound I comprises about 69 mg BID. In an embodiment, the therapeutically effective amount of Compound I comprises about 70 mg BID. In an embodiment, the therapeutically effective amount of Compound I comprises about 71 mg BID. In an embodiment, the therapeutically effective amount of Compound I comprises about 72 mg BID. In an embodiment, the therapeutically effective amount of Compound I comprises about 73 mg BID. In an embodiment, the therapeutically effective amount of Compound I comprises about 74 mg BID. In an embodiment, the therapeutically effective amount of Compound I comprises about 75 mg BID. In an embodiment, the therapeutically effective amount of Compound I comprises about 76 mg BID. In an embodiment, the therapeutically effective amount of Compound I comprises about 77 mg BID. In an embodiment, the therapeutically effective amount of Compound I comprises about 78 mg BID. In an embodiment, the therapeutically effective amount of Compound I comprises about 79 mg BID. In an embodiment, the therapeutically effective amount of Compound I comprises about 80 mg BID. In preferred embodiments, the therapeutically effective amount of Compound I comprises about 5 mg BID, about 10 mg BID, about 15 mg BID, about 25 mg BID, or 40 mg BID. In an embodiment, the therapeutically effective amount of Compound I comprises about 5 mg BID.

In any embodiment, 5 mg TID of Compound I is administered to a human subject having an inflammatory condition disclosed herein. In any embodiment, 10 mg TID of Compound I is administered to a human subject having an inflammatory condition disclosed herein. In any embodiment, 15 mg TID of Compound I is administered to a human subject having an inflammatory condition disclosed herein. In any embodiment, 25 mg TID of Compound I is administered to a human subject having an inflammatory condition disclosed herein. In any embodiment, 30 mg TID of Compound I is administered to a human subject having an inflammatory condition disclosed herein. In any embodiment, 40 mg TID of Compound I is administered to a human subject having an inflammatory condition disclosed herein.

In any embodiment, the therapeutically effective amount of Compound I is about 1 mg TID to about 100 mg TID or any amount in between. In any embodiment, the therapeutically effective amount of Compound I is about 2 mg TID to about 50 mg TID. In any embodiment, the therapeutically effective amount of Compound I is about 3 mg TID to about 50 mg TID. In any embodiment, the therapeutically effective amount of Compound I is about 4 mg TID to about 50 mg TID. In any embodiment, the therapeutically effective amount of Compound I is about 5 mg TID to about 50 mg TID. In any embodiment, the therapeutically effective amount of Compound I is about 10 mg TID to about 50 mg TID. In any embodiment, the therapeutically effective amount of Compound I is about 15 mg TID to about 50 mg TID. In any embodiment, the therapeutically effective amount of Compound I is about 20 mg TID to about 50 mg TID. In any embodiment, the therapeutically effective amount of Compound I is about 25 mg TID to about 50 mg TID. In any embodiment, the therapeutically effective amount of Compound I is about 30 mg TID to about 50 mg TID. In any embodiment, the therapeutically effective amount of Compound I is about 35 mg TID to about 100 mg TID. In any embodiment, the therapeutically effective amount of Compound I is about 40 mg TID to about 50 mg TID. In any embodiment, the therapeutically effective amount of Compound I is about 45 mg TID to about 50 mg TID. In any embodiment, the therapeutically effective amount of Compound I is about 1 mg TID to about 45 mg TID. In any embodiment, the therapeutically effective amount of Compound I is about 1 mg TID to about 40 mg TID. In any embodiment, the therapeutically effective amount of Compound I is about 1 mg TID to about 35 mg TID. In any embodiment, the therapeutically effective amount of Compound I is about 1 mg TID to about 30 mg TID. In any embodiment, the therapeutically effective amount of Compound I is about 1 mg TID to about 25 mg TID. In any embodiment, the therapeutically effective amount of Compound I is about 1 mg TID to about 20 mg TID. In any embodiment, the therapeutically effective amount of Compound I is about 1 mg TID to about 15 mg TID. In any embodiment, the therapeutically effective amount of Compound I is about 1 mg TID to about 10 mg TID. In any embodiment, the therapeutically effective amount of Compound I is about 1 mg TID to about 5 mg TID. In an embodiment the therapeutically effective amount of Compound I is about 1 mg TID to about 50 mg TID. In an embodiment the therapeutically effective amount of Compound I is about 2.5 mg TID to about 20 mg TID.

In an embodiment, the therapeutically effective amount of Compound I comprises about 1 mg TID. In an embodiment, the therapeutically effective amount of Compound I comprises about 2 mg TID. In an embodiment, the therapeutically effective amount of Compound I comprises about 2.5 mg TID. In an embodiment, the therapeutically effective amount of Compound I comprises about 3 mg TID. In an embodiment, the therapeutically effective amount of Compound I comprises about 4 mg TID. In an embodiment, the therapeutically effective amount of Compound I comprises about 5 mg TID. In an embodiment, the therapeutically effective amount of Compound I comprises about 6 mg TID. In an embodiment, the therapeutically effective amount of Compound I comprises about 7 mg TID. In an embodiment, the therapeutically effective amount of Compound I comprises about 8 mg TID. In an embodiment, the therapeutically effective amount of Compound I comprises about 9 mg TID. In an embodiment, the therapeutically effective amount of Compound I comprises about 10 mg TID In an embodiment, the therapeutically effective amount of Compound I comprises about 11 mg TID In an embodiment, the therapeutically effective amount of Compound I comprises about 12 mg TID In an embodiment, the therapeutically effective amount of Compound I comprises about 13 mg TID In an embodiment, the therapeutically effective amount of Compound I comprises about 14 mg TID In an embodiment, the therapeutically effective amount of Compound I comprises about 15 mg TID In an embodiment, the therapeutically effective amount of Compound I comprises about 16 mg TID In an embodiment, the therapeutically effective amount of Compound I comprises about 17 mg TID In an embodiment, the therapeutically effective amount of Compound I comprises about 18 mg TID In an embodiment, the therapeutically effective amount of Compound I comprises about 19 mg TID In an embodiment, the therapeutically effective amount of Compound I comprises about 20 mg TID In an embodiment, the therapeutically effective amount of Compound I comprises about 21 mg TID In an embodiment, the therapeutically effective amount of Compound I comprises about 22 mg TID In an embodiment, the therapeutically effective amount of Compound I comprises about 23 mg TID In an embodiment, the therapeutically effective amount of Compound I comprises about 24 mg TID In an embodiment, the therapeutically effective amount of Compound I comprises about mg TID In an embodiment, the therapeutically effective amount of Compound I comprises about 26 mg TID In an embodiment, the therapeutically effective amount of Compound I comprises about 27 mg TID In an embodiment, the therapeutically effective amount of Compound I comprises about 28 mg TID In an embodiment, the therapeutically effective amount of Compound I comprises about 29 mg TID In an embodiment, the therapeutically effective amount of Compound I comprises about 30 mg TID In an embodiment, the therapeutically effective amount of Compound I comprises about 31 mg TID In an embodiment, the therapeutically effective amount of Compound I comprises about 32 mg TID In an embodiment, the therapeutically effective amount of Compound I comprises about 33 mg TID In an embodiment, the therapeutically effective amount of Compound I comprises about 34 mg TID In an embodiment, the therapeutically effective amount of Compound I comprises about 35 mg TID In an embodiment, the therapeutically effective amount of Compound I comprises about 36 mg TID In an embodiment, the therapeutically effective amount of Compound I comprises about 37 mg TID In an embodiment, the therapeutically effective amount of Compound I comprises about 38 mg TID In an embodiment, the therapeutically effective amount of Compound I comprises about 39 mg TID In an embodiment, the therapeutically effective amount of Compound I comprises about 40 mg TID In an embodiment, the therapeutically effective amount of Compound I comprises about 41 mg TID In an embodiment, the therapeutically effective amount of Compound I comprises about 42 mg TID In an embodiment, the therapeutically effective amount of Compound I comprises about 43 mg TID In an embodiment, the therapeutically effective amount of Compound I comprises about 44 mg TID In an embodiment, the therapeutically effective amount of Compound I comprises about 45 mg TID In an embodiment, the therapeutically effective amount of Compound I comprises about 46 mg TID In an embodiment, the therapeutically effective amount of Compound I comprises about 47 mg TID In an embodiment, the therapeutically effective amount of Compound I comprises about 48 mg TID In an embodiment, the therapeutically effective amount of Compound I comprises about 49 mg TID In an embodiment, the therapeutically effective amount of Compound I comprises about 50 mg TID In an embodiment, the therapeutically effective amount of Compound I comprises about 51 mg TID In an embodiment, the therapeutically effective amount of Compound I comprises about 52 mg TID In an embodiment, the therapeutically effective amount of Compound I comprises about 53 mg TID In an embodiment, the therapeutically effective amount of Compound I comprises about 54 mg TID In an embodiment, the therapeutically effective amount of Compound I comprises about 55 mg TID In an embodiment, the therapeutically effective amount of Compound I comprises about 56 mg TID In an embodiment, the therapeutically effective amount of Compound I comprises about 57 mg TID In an embodiment, the therapeutically effective amount of Compound I comprises about 58 mg TID In an embodiment, the therapeutically effective amount of Compound I comprises about 59 mg TID In an embodiment, the therapeutically effective amount of Compound I comprises about 60 mg TID In an embodiment, the therapeutically effective amount of Compound I comprises about 61 mg TID In an embodiment, the therapeutically effective amount of Compound I comprises about 62 mg TID In an embodiment, the therapeutically effective amount of Compound I comprises about 63 mg TID In an embodiment, the therapeutically effective amount of Compound I comprises about mg TID In an embodiment, the therapeutically effective amount of Compound I comprises about mg TID In an embodiment, the therapeutically effective amount of Compound I comprises about 66 mg TID In an embodiment, the therapeutically effective amount of Compound I comprises about 67 mg TID In an embodiment, the therapeutically effective amount of Compound I comprises about 68 mg TID In an embodiment, the therapeutically effective amount of Compound I comprises about 69 mg TID In an embodiment, the therapeutically effective amount of Compound I comprises about 70 mg TID In an embodiment, the therapeutically effective amount of Compound I comprises about 71 mg TID In an embodiment, the therapeutically effective amount of Compound I comprises about 72 mg TID In an embodiment, the therapeutically effective amount of Compound I comprises about 73 mg TID In an embodiment, the therapeutically effective amount of Compound I comprises about 74 mg TID In an embodiment, the therapeutically effective amount of Compound I comprises about 75 mg TID In an embodiment, the therapeutically effective amount of Compound I comprises about 76 mg TID In an embodiment, the therapeutically effective amount of Compound I comprises about 77 mg TID In an embodiment, the therapeutically effective amount of Compound I comprises about 78 mg TID In an embodiment, the therapeutically effective amount of Compound I comprises about 79 mg TID In an embodiment, the therapeutically effective amount of Compound I comprises about 80 mg TID. In preferred embodiments, the therapeutically effective amount of Compound I comprises about 5 mg TID, about 10 mg TID, about 15 mg TID, about 25 mg TID, or 40 mg TID.

In any embodiment, the oral dose of Compound I that is administered to the human subject in accordance with the methods disclosed herein comprises a free base. In any embodiment, the oral dose of Compound I that is administered to the human subject in accordance with the methods disclosed herein comprises a pharmaceutically acceptable salt.

In any embodiment a human subject having an inflammatory condition is administered about 1 mg/day to about 100 mg/day of Compound I.

In any embodiment a human subject having an inflammatory condition is administered about 1 mg BID to about 100 mg BID of Compound I.

In any embodiment a human subject having an inflammatory condition is administered about 1 mg TID to about 100 mg TID of Compound I.

In any embodiment a human subject having an inflammatory condition is administered about 10 mg/day to about 80 mg/day of Compound I.

In any embodiment a human subject having an inflammatory condition is administered about 5 mg BID to about 40 mg BID of Compound I.

The dosage administered is a therapeutically effective amount of the composition sufficient to result in amelioration of a symptom or symptoms, and can vary depending upon known factors such as the pharmacodynamic characteristics of the active ingredient and its mode of administration; age, sex, health and weight of the subject; nature and extent of symptoms; kind of concurrent treatment, frequency of treatment and the effect desired.

In some embodiments, the oral composition comprising Compound I as described herein can be administered to the subject once (e.g., as a single dose or application). In some embodiments, the oral composition of embodiments herein is administered at least once daily, such as at least two, three or four times daily. In some embodiments, the oral composition of embodiments herein may be administered daily, twice daily, three times daily, weekly, twice weekly, every two weeks, every three weeks, monthly, as needed, or as otherwise directed by a physician. The oral composition of embodiments herein may be administered at any interval to achieve the therapeutically desired effect, e.g., induction or maintenance of remission, prevention or relief of a symptom or symptoms. In some embodiments, the oral composition of embodiments herein may be administered to a subject for a period of 1, 2, 3, 4, 5, 6 days, about a week, about two weeks, about three weeks, about four weeks, about five weeks, about six weeks, about two months, about three months, about four months, about five months, about six months, or a range of any two of these values. In some embodiments, treatment may be continued for at least a week, a month, a year, or as otherwise directed by a physician. In some embodiments, treatment may extend over multiple years, the duration of disease, or the lifetime of the subject. In some embodiments, the oral composition of embodiments herein can be administered once or twice daily to a subject in need thereof for a period of about two to about twenty-eight days, or from about seven to about ten days. The oral composition of embodiments herein can also be administered once, twice, or three times daily to a subject for a period of 1, 2, 3, 4, 5, 6, 7, 8, 9, 10, 11, 12 times per year, or a combination thereof.

In some embodiments the oral composition comprising Compound I as described herein is administered before, after, or with a meal. In some embodiments the oral composition described herein is administered before, after, or with a high fat meal. In some embodiments, the oral composition described herein is administered before, after, or with a standardized high-fat meal. In some embodiments, the high-fat meal is a high-caloric, high-fat meal. In some embodiments, the high-fat meal follows the FDA guidance on a high-fat meal. In some embodiments, the high-calorie, high-fat meal follows the FDA guidance on a high-fat and high-calorie meal. In some embodiments, the high-fat meal comprises a fat content of about 50% or greater of total caloric content of the meal. See U.S. Department of Health and Human Services Food and Drug Administration Center for Drug Evaluation and Research, (2002). Guidance for Industry: Food-Effect Bioavailability and Fed Bioequivalence Studies. Office of Training and Communications Division of Drug Information, HFD-240. In some embodiments, the high-calorie, high-fat meal comprises a fat content of at least 50% of total caloric content of the meal and a total of about 800 to about 1000 kilocalorie content.

In some embodiments the oral composition described herein is administered following an overnight fast. In some embodiments, the overnight fast is at least about 6 hours, at least about 7 hours, at least about 8 hours, at least about 9 hours, or at least about 10 hours. For example, the oral composition described herein may be administered following a high-fat meal, high-calorie meal following an overnight fast of at least 10 hours.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

The disclosure is further illustrated by the following examples.

### EXAMPLES

### Example 1: A Phase 1 Placebo-Controlled, Randomized, Observer-Blind, Single Ascending Dose Study to Investigate the Safety, Tolerability, Pharmacokinetics, and Pharmacodynamics of Compound I In Healthy Subjects

Compound I, an orally available small molecule covalent inhibitor of Interleukin 2-Inducible T cell kinase (ITK; also known as EMT or TSK), tyrosine protein kinase TXK (also known as Resting Lymphocyte Kinase or RLK) and Janus kinase (JAK) 3 for treatment of T cell-mediated autoimmune disease. ITK and TXK are two of the Tec family of tyrosine kinases which consists of five family members: Tec, Bruton's tyrosine kinase (BTK), bone marrow-expressed kinase (BMX), TXK, and ITK. These kinases are central to the regulation of hematopoietic cell biology and more specifically to the development and activity of lymphocytes and myeloid cells. The roles of ITK and TXK in T cell function have been delineated through genetic knockdown/kinase inactivation of these genes in rodents. JAK3 is a key signaling molecule downstream of numerous cytokines—the common γ chain family of cytokine receptors—which include interleukin (IL) 2, 4, 7, 9, 15, and 21. These cytokines are involved in the autoimmune response. Inhibiting ITK, TXK, and JAK 3 may be a specific and effective way to inhibit the activation and survival of T cells.

The first therapeutic indication for human testing is moderate-to-severe plaque psoriasis. Additional therapeutic indications of interest are rheumatoid arthritis (RA), hidradenitis suppurativa (HS), psoriatic arthritis (PSoA), ulcerative colitis (UC), and Crohn's disease.

Study Rational—This study provided an assessment of the safety, tolerability, PK, and pharmacodynamics (PD) of Compound I after administration of ascending single oral doses to healthy subjects. The study also assessed the effect of formulation on the PK of Compound I following the administration of single oral dose of Compound I to provide a dosing guidance in upcoming planned studies in moderate-to-severe plaque psoriasis (MSPP). The effect of food on the PK of Compound I following a single oral dose was also assessed.

Primary Objectives—The safety, tolerability, and PK profile of Compound I was assessed following single oral doses of Compound I in healthy subjects.

Primary Endpoints—Number and percentage of adverse events (AEs) and serious adverse events (SAEs); mean change from baseline in laboratory values, vital signs, and electrocardiograms (ECGs) abnormalities.

Secondary Objectives—The effect of food on the PK of Compound I following administration of a single oral dose of Compound I was assessed. The effect of formulation on the PK of Compound I following administration of a single oral dose of Compound I was assessed. The PD response to Compound I following single oral dose of Compound I was assessed.

Secondary Endpoints—Compound I plasma concentrations. Mean change from baseline in ex vivo stimulated pSTAT5 and pSTAT1 (signal transducer and activator of transcription); and in ex vivo stimulated IFNγ mRNA (interferon gamma messenger ribonucleic acid).

### Overall Study Design and Plan

Type of Study—This was an FIH, randomized, observer-blind, placebo-controlled study designed to evaluate the safety, tolerability, PK, and PD of Compound I in healthy subjects. The study consisted of: single ascending dose (SAD) cohorts (1-80 mg/day) plus formulation bridging and food effect (FE) cohorts.

Approximately 64 male and female subjects were enrolled into 8 planned cohorts in the SAD study. In each cohort, a total of 8 subjects were randomized to receive a single oral capsule dose of Compound I (n=6) or placebo (n=2) in the morning with approximately 240 mL of water after an overnight fast of at least 8 hours.

Dose levels are listed in Table 1.

Cohort 4 was an FE (food effect) cohort where subjects received Compound I capsules or placebo, fasted on Day 1 and Compound I or placebo after a high-fat, high-calorie breakfast on Day 8 after a 7-day washout period.

Cohort 5 was a formulation bridging cohort where subjects will receive Compound I capsules or placebo on Day 1 and Compound I tablets or placebo on Day 8 after a 7-day washout period.

The dosage form of Compound I in Cohorts 6, 7, and 8 were capsules and the subjects will receive a single oral dose of Compound I (n=6) capsules or placebo (n=2) on Day 1 after an overnight fast of at least 8 hours.

TABLE 1
Does Levels of Compound I - SAD

| Cohort | Number of Subjects | Planned Single Dose Level (mg) |
|---|---|---|
| 1 | 8 | 1 |
| 2 | 8 | 3 |
| 3 | 8 | 5 |
| 4^{a} | 8 | 15^{a} |
| 5^{b, c} | 8 | 25^{c} |
| 6 | 8 | 25 |
| 7 | 8 | 50 |
| 8 | 8 | 80 |

^{a}Cohort 4 (FE) will be administered a 15 mg dose of Compound I capsules or placebo, fasted on Day 1 and after a high-fat, high-calorie breakfast on Day 8. In the event that the kinetic profile is different than the anticipated profile, the FE may be studied in another cohort.
^{b}Cohort 5 (formulation bridging) will be administered a TBD (proposed) dose of Compound I capsule or placebo on Day 1 and a matching dose of Compound I tablet or placebo on Day 8. In the event that the regulatory notification is not submitted for Compound I tablets, Cohort 5 will only be administered Compound I capsules.
^{c}Cohorts 5 onwards doses will be increased as per the recommendation of the SRC. The maximum increase between cohorts will be 2 fold increase. The target exposure level for the last dose level chosen in this study is based upon NOAEL (no observed adverse effect level) of 983 ng*hr/mL.

### Data Summary

PK was linear and absorption was fast. This shows that Compound I has a favorable PK profile up to 80 mg single dose.

Exposure was dose-proportional (Linear Cₘₐₓ and AUC).

Absorption was fast (Tmax 0.7-1.2 h).

Absorption was fast (Tmax 0.7-1.2 h)

Half-life ranges 1.5-2.5 h.

Within cohort exposure variability CV ranges from 21% to 64%.

Food-Effect & Formulation—A high-calorie, high-fat meal reduces Cmax by 31%, and delays Tmax from 1 hr to 5 hrs, but it had no significant effect on AUC, bioavailability, or T1/2 of a single 15 mg dose. No significant difference in PK parameters from a 25 mg single dose between capsule formulation (SAD) and tablet formulation.

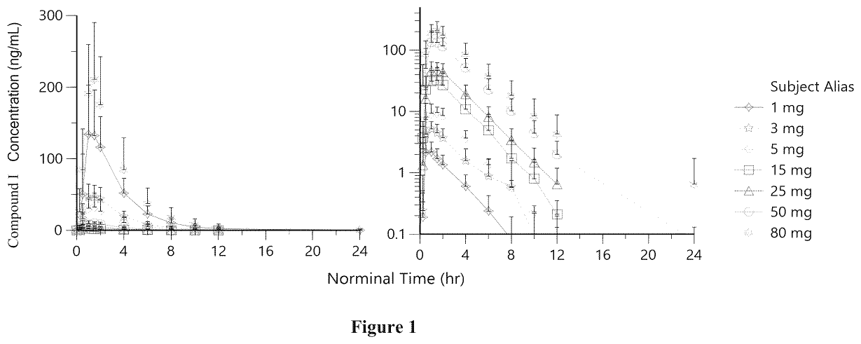

Observed Human Compound I PK (1-80 mg, Preliminary PK Parameters) are shown in FIG. 1 and Table 2.

TABLE 2

| | | | AUC_{0-∞} | | | | AUC₀₋₂₄ |
|---|---|---|---|---|---|---|---|
| Dose | Cₘₐₓ | Tₘₐₓ | (hr*ng/ | T_{1/2} | CL/F | Vz/F | (hr*ng/ |
| (mg/day) | (ng/mL) | (hr) | mL) | (hr) | (L/hr) | (L) | mL) |
| 1 | 2.71 | 0.674 | 5.95 | 1.51 | 168 | 384 | 5.95 |
| 3 | 5.9 | 0.757 | 15.8 | 1.53 | 190 | 419 | 15.4 |
| 5 | 11.9 | 1.07 | 33.5 | 1.53 | 149 | 331 | 33.1 |
| 15 | 31.5 | 1.09 | 96.7 | 1.61 | 155 | 360 | 95.7 |
| 25 | 47.0 | 1.35 | 154 | 1.57 | 162 | 366 | 153 |
| 50 | 143 | 1.24 | 428 | 1.84 | 117 | 310 | 426 |
| 80 | 196 | 1.22 | 676 | 2.53 | 118 | 432 | 675 |

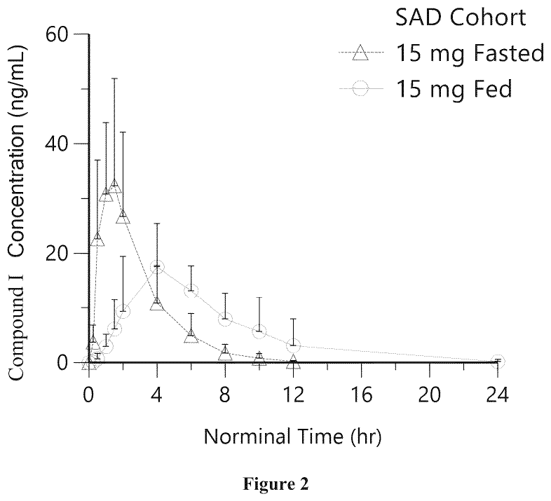

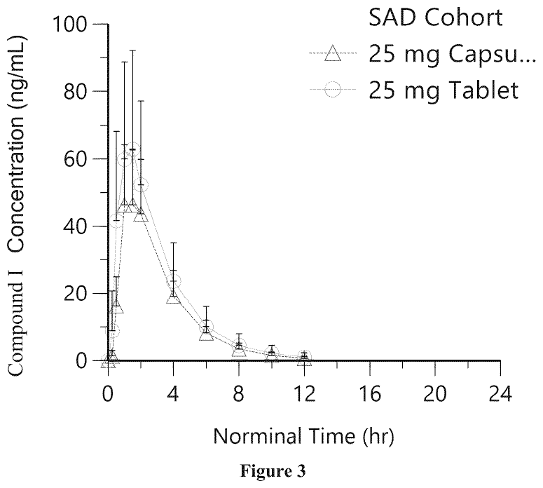

Food and formulation effect on AUC Data are shown in FIG. 2 (15 mg), FIG. 3 (25 mg), and Table 3.

TABLE 3

| | | | AUC_{0-∞} | | | | AUC₀₋₂₄ |
|---|---|---|---|---|---|---|---|
| Dose | Cₘₐₓ | Tₘₐₓ | (hr*ng/ | T_{1/2} | CL/F | Vz/F | (hr*ng/ |
| (mg/day) | (ng/mL) | (hr) | mL) | (hr) | (L/hr) | (L) | mL) |
| 15, Fasted | 31.5 | 1.09 | 96.7 | 1.61 | 155 | 360 | 95.7 |
| 15, Fed | 17.6 | 4.99 | 93.9 | 1.73 | 160 | 399 | 105 |
| 25, Capsule | 47.0 | 1.35 | 154 | 1.57 | 162 | 366 | 153 |
| 25, Tablet | 63.6 | 1.07 | 199 | 1.75 | 126 | 317 | 197 |

SAD Exploratory PD Efforts—PD markers of both ITK (αCD3/αCD28 induced IL2 and IFNγ mRNA) and JAK3 (IL2 induced pSTAT5) activation were dose-dependently inhibited by Compound I. Simultaneous activation of the ITK and JAK3 pathways (αCD3/IL-15-induced IFNγ protein production) was dose-dependently inhibited by Compound I. Near complete inhibition of the dual ITK and JAK3-stimulated IFNγ protein production was observed in the higher dose cohorts with >50% inhibition maintained for 12 hours.

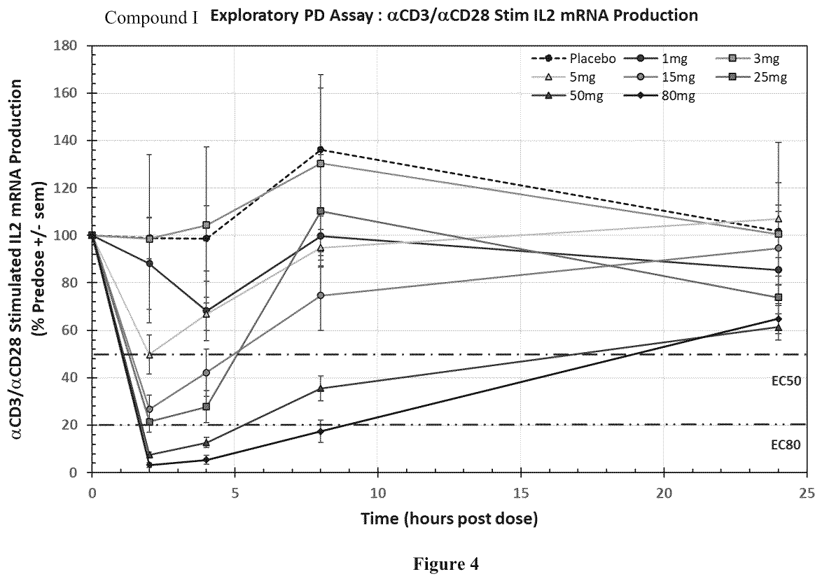

αCD3/αCD28 stimulated IL2 mRNA production is depicted in FIG. 4.

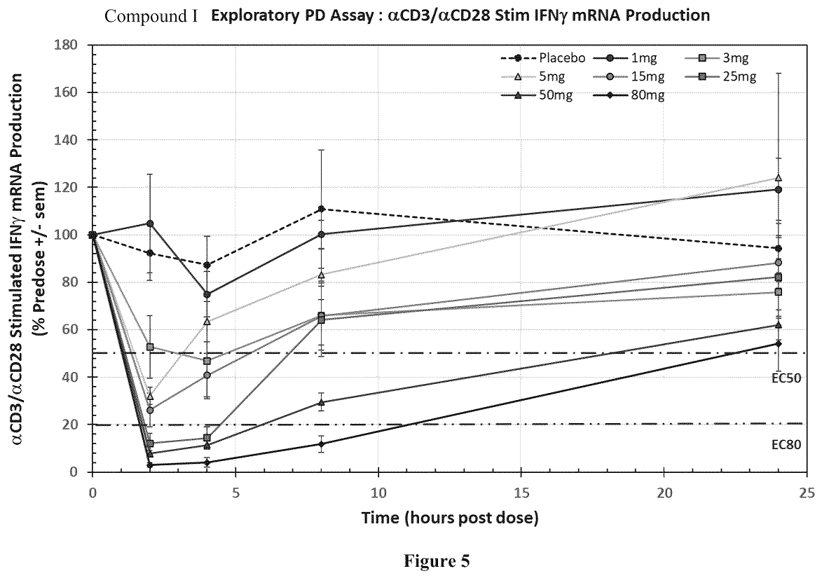

αCD3/αCD28 stimulated IFNγ mRNA production is depicted in FIG. 5.

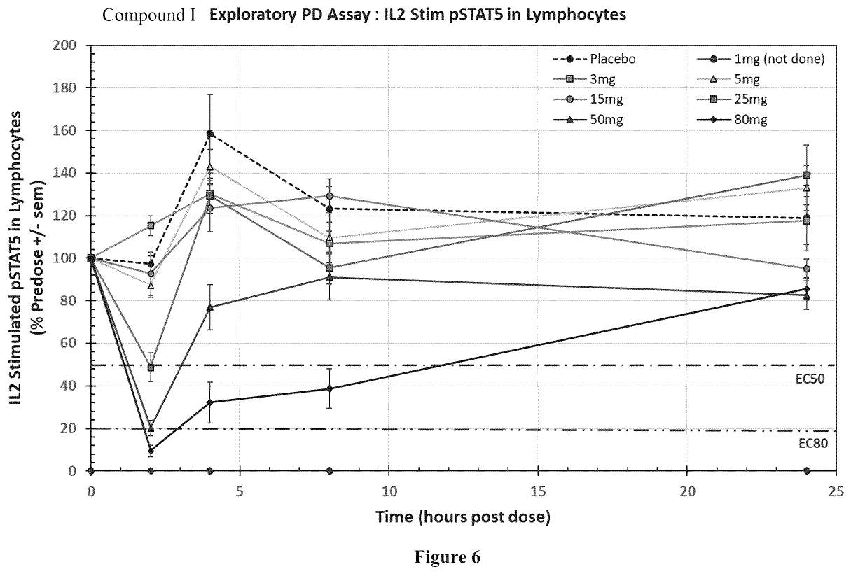

IL2 stimulated pSTAT5 in Lymphocytes is depicted in FIG. 6.

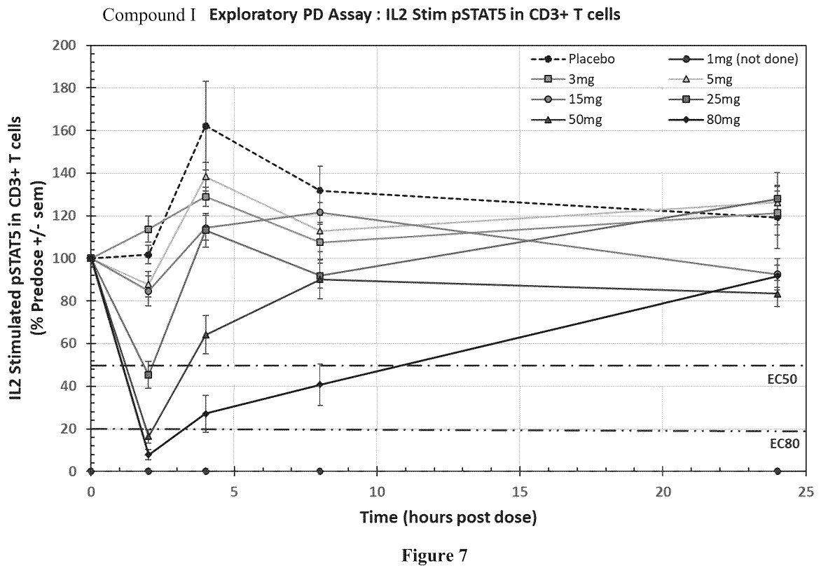

IL2 stimulated pSTAT5 in CD3+ T Cells is depicted in FIG. 7.

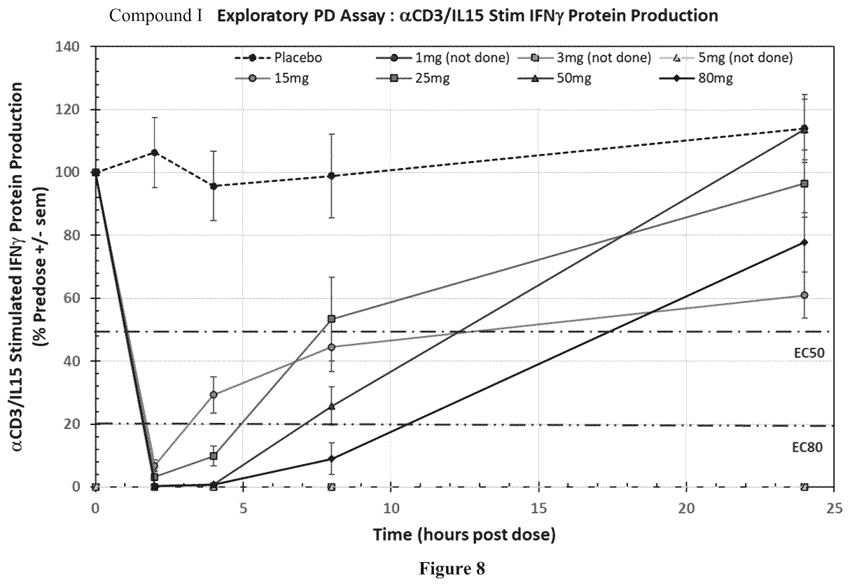

αCD3/IL15 stimulated IFNγ protein production is depicted in FIG. 8.

Safety Data Summary—Safety data was very re-assuring: 17 TEAEs; 14 in Compound I treated subjects and 3 in placebo subjects, all 14 TEAEs (13 subjects) in Compound I treated subjects were mild and transient. No clinically meaningful changes in WBC count were seen during treatment and at follow-up. Transient increase in transaminases (1 subject) and CK (2 subjects) were seen at follow-up in the highest dose group (80 mg) and all had resolved on subsequent follow-up.

Results: Sixty-three subjects completed the SAD study. There were no deaths, serious AEs, or discontinuations due to AEs. Plasma concentrations of Compound I increased in a dose-dependent manner. Peak concentrations were reached within 2 hours after dosing. Compound I caused dose- and time-dependent modulation of all PD biomarker readouts (αCD3/αCD28 stimulated interleukin IL2 and interferon IFNγ mRNA levels, IL15 stimulated and αCD3/IL-15 stimulated IFNγ protein production). Near-complete inhibition of biomarker readouts was observed 2 hours post-Compound I treatment at doses of 15 mg to 80 mg.

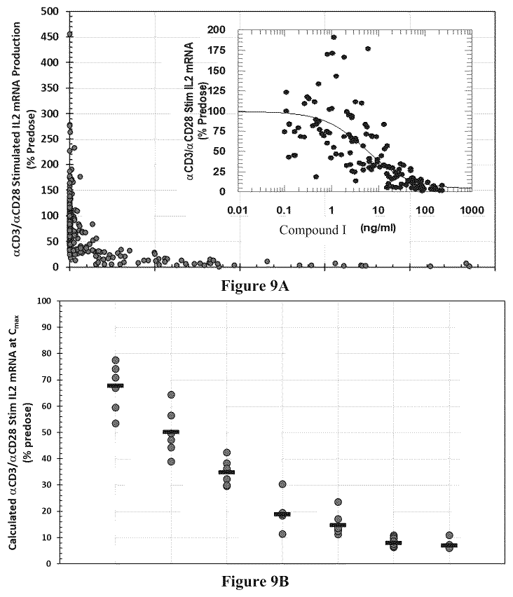

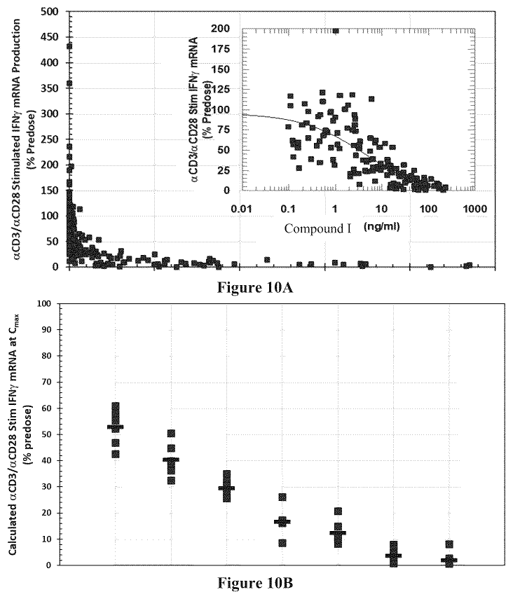

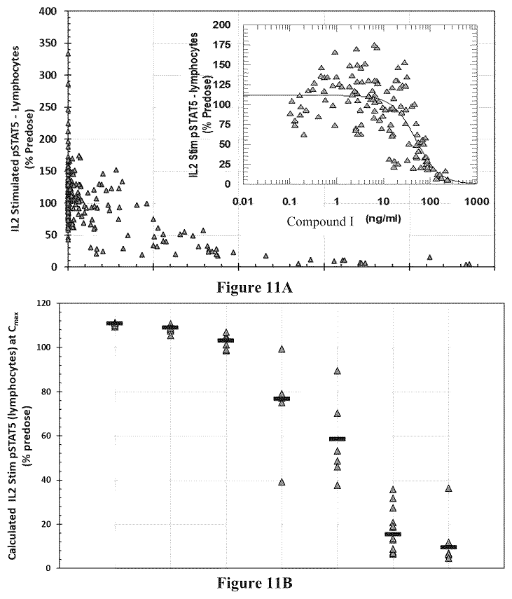

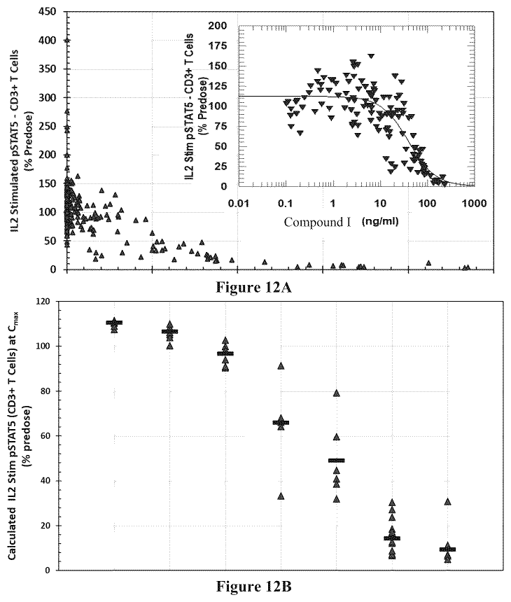

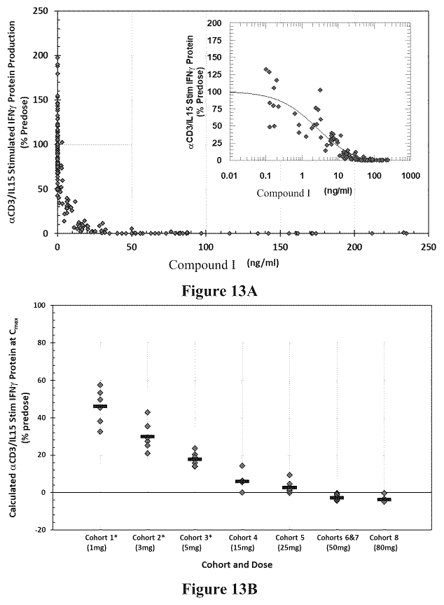

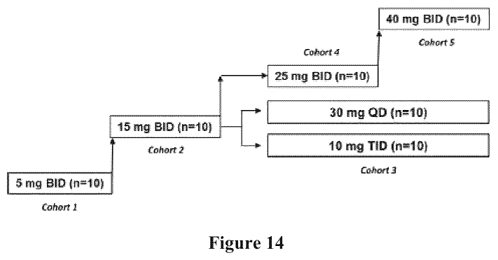

Summary of SAD data: FIGS. 4-8 and 9-13 show the SAD dose response, SAD exposure response, and SAD calculated response at Cₘₐₓ by dose respectively for ITK (IL2 mRNA), ITK (IFNγ mRNA), JAK3 (pSTAT5, lymph), JAK3 (pSTAT5, CD3′ T), and ITK & JAK (IFNγ protein). Clinical and pre-clinical IC₅₀ data for ITK (IL2 mRNA), ITK (IFNγ mRNA), JAK3 (pSTAT5, lymph), JAK3 (pSTAT5, CD3⁺ T), and ITK & JAK (IFNγ protein) from the SAD study is shown in table 4.

TABLE 4

| | | Pre-clinical |
|---|---|---|
| Target | Clinical EC₅₀ (nM) | EC₅₀ (nM) |
| ITK (IL2 mRNA) | 15.2 | 21.3 ± 4.3 |
| ITK (IFNγ mRNA) | 12.7 | 17.3 ± 3.0 |
| JAK3 (pSTAT5, lymph) | 137.3 | 136.1 ± 51.2 |
| JAK3 (pSTAT5, CD3⁺ T) | 109.6 | 140.1 ± 49.3 |
| ITK & JAK (IFNγ protein) | 7.3 | 10.2 ± 4.1 |

Conclusions: Compound I was safe and well tolerated and resulted in dose-dependent increases in plasma Compound I and PD effects consistent with the inhibition of ITK and JAK3.

### Example 2: A Phase 1 Placebo-Controlled, Randomized, Investigator and Participant-Blind, Sponsor-Unblinded Combined Multiple Ascending Dose Study to Investigate the Safety, Tolerability, Pharmacokinetics, and Pharmacodynamics of Compound I in Healthy Participants

Study Rationale—This study provided an assessment of the safety, tolerability, pharmacokinetics (PK), and pharmacodynamics (PD) of Compound I after administration of ascending multiple oral doses to healthy participants. The study also included a comparison of the effect of delivering a daily dose of 30 mg as one fixed dose (30 mg once daily [QD]) vs a split dose (10 mg three times daily [TID]) on the PK of Compound I to provide dosing guidance in upcoming planned studies in ulcerative colitis (UC).

### Objectives and Endpoints

Primary Objectives—To assess the safety and tolerability of Compound I following multiple oral doses of Compound I in healthy participants.

Primary Endpoints—Number and percent of adverse events (AEs) and serious adverse events (SAEs); mean change from baseline in laboratory values, vital signs, and electrocardiograms (ECGs).

Secondary Objectives—To explore the pharmacokinetic (PK) profile of Compound I following multiple oral doses of Compound I in healthy participants.

Secondary Endpoints—Compound I plasma concentrations and PK parameters: AUC0-tau, AUC0-inf, Ctrough, Cmax, tmax, Kel, t1/2, CLss/F, Vss/F, Rae.

Exploratory Objectives—To explore the pharmacodynamic (PD) response to Compound I following multiple oral doses of Compound I in healthy participants.

Exploratory Endpoints—Mean change from baseline in cellular, extracellular (secreted), and/or message markers of inflammation.

### Study Design

Overall Design—This was a randomized, Investigator and participant-blind. Sponsor-unblinded, placebo-controlled study designed to evaluate the safety, tolerability, PK, and PD of Compound I in healthy participants. The study consisted of multiple cohorts assessing ascending doses of Compound I.

Approximately 60 participants were enrolled into 5 cohorts. At each dose level/cohort, a total of 10 participants were randomized to receive 2 weeks of oral doses of Compound I (n=8) or placebo (n=2). In Cohort 3, 20 participants were randomized to receive multiple oral doses of Compound I QD (n=8), Compound I TID (n=8), placebo QD (n=2), or placebo TID (n=2). Cohort 3 proceed in parallel with Cohorts 4 and 5 and did not require a dose escalation meeting.

The study started with a planned dose of 5 mg twice daily (BID). Planned dose levels and dosing frequency are listed in tables 5 and 6 below. Escalation to the next higher cohort was dependent on safety and PK data from all participants in the previous cohorts and was based on data from at least 6 participants up to at least 48 hours after the last dose. In no instance did the dose escalation steps exceed 3-fold the prior dose level.

TABLE 5
Dose Escalation Steps for Multiple Ascending Doses

| | | Planned Dose |
|---|---|---|
| | Number of | Level (mg) |
| Cohort | Participants | for 14 days^{a} |
| 1 | 10 | 5 mg BID |
| 2 | 10 | 15 mg BID |
| 3 (in parallel with Cohorts 4 | 20 | 30 mg QD and |
| and 5; not part of dose escalation) | | 10 mg TID |
| 4 | 10 | 25 mg BID |
| 5 | 10 | 40 mg BID |

BID: Twice daily;
QD: Once daily;
TID: Three times daily
^{a}The final dose was administered on the morning of Day 15

TABLE 6
Dose Escalation Steps for Multiple Ascending Doses

| Intervention | | |
|---|---|---|
| Label | Compound I | Placebo |
| Type | Drug | Placebo |
| Dose | Tablet | Tablet |
| Formulation | | |
| Unit Dose | 5 mg | 0 mg |
| Strength(s) | | |
| Dosage | 5 mg BID | |
| Level(s) | or 15 mg | 0 mg QD or |
| | BID or 30 | 0 mg BID or |
| | mg QD or | 0 mg TID |
| | 10 mg TID | |
| | or 25 mg | |
| | BID or 40 | |
| | mg BID | |
| Route of | Oral | Oral |
| Administration | | |
| Sourcing | Provided by the Sponsor | Provided by the Sponsor |
| Packaging and | 30 cc HDPE bottle with foil sealed | 30 cc HDPE bottle with foil sealed |
| Labeling | polypropylene CR cap Each bottle | polypropylene CR cap. Each bottle |
| | will be labeled as required per | will be labeled as required per |
| | country requirement. | country requirement. |

BID = Twice daily;
CR = Child-resistant;
HDPE = High Density Polyethylene;
QD = Once daily;
TID = Three times daily

Results: 57 subjects completed the MAD study. There were no deaths, serious AEs, or discontinuations due to AEs. Plasma concentrations of Compound I increased in a dose-dependent manner. Peak concentrations were reached within 2 hours after dosing. Compound I caused dose- and time-dependent modulation of all PD biomarker readouts (αCD3/αCD28 stimulated interleukin IL2 and interferon IFNγ mRNA levels, IL15 stimulated and αCD3/IL-15 stimulated IFNγ protein production). Near-complete inhibition of biomarker readouts was observed 2 hours post-Compound I treatment at doses of 15 mg to 80 mg.

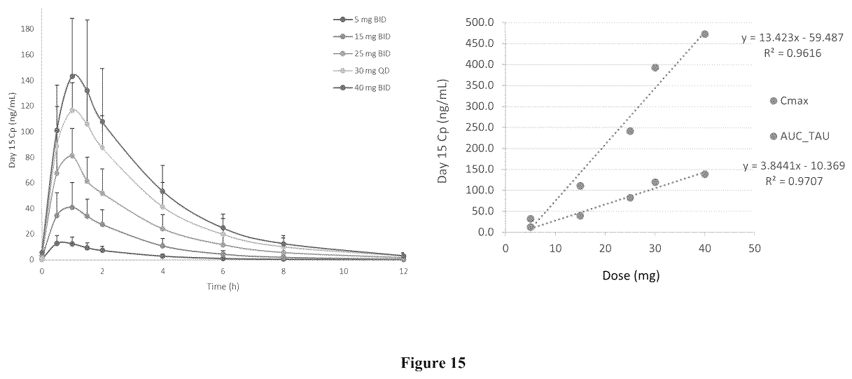

Summary of MAD data: Table 7 shows the safety profile for Compound I from the MAD study. FIGS. 15-18 and table 8 show pharmacodynamic and pharmacokinetic data for oral administration of Compound I from the MAD study.

Table 7 shows that Compound I is safe and tolerable up to 2 weeks administration in HV.1 adverse event (AE) of Special Interest (shingles) at highest dose tested consistent with known properties of JAK inhibitors.

TABLE 7

| Events | 5 mg BID | 15 mg BID | 25 mg BID | 40 mg BID | 30 mg QD | 10 mg TID |
|---|---|---|---|---|---|---|
| AEs | 9 | 0 | 4 | 4 | 3 | 3 |
| AE of | 3 | 0 | 1 | 1 | 0 | 0 |
| Special | | | | | | |
| Interest | | | | | | |
| ≥Grade 2 | 1 | 0 | 0 | 1 | 0 | 0 |
| Notable | 5 mg BID | | Placebo | 40 mg BID | | |
| events | 2 subjects | | 1 subject | 1 subject | | |
| | with single | | developed | developed single | | |
| | episode of | | elevated CK | dermatomal zoster | | |
| | transient | | after surreptitious | rash 1 week after | | |
| | diarrhea; | | exercising; | dosing ended. | | |
| | Placebo | | resolving after | No PMH to suggest | | |
| | 1 subject | | discharge | immunodeficiency; | | |
| | with benign | | | no history of | | |
| | ethnic | | | zoster in past | | |
| | neutropenia | | | | | |

FIG. 15 and table 8 show the pharmacokinetics of Compound I at several doses. The geometric mean exposure (Cₘₐₓ and AUC) increases slightly greater than dose-proportional. To reach HWB dual stim EC50, human Compound I Cₚ>=3.47 (MAD PD data) or 4.19 (in vitro report) ng/mL. At 15 mg BID and steady-state 6 hours post-dose. Cₚ=4.3 ng/mL Duration above EC5: 12 hours/day. At 25 mg BID and steady-state 8 hours post-dose, =5.7 ng/mL Duration above EC50: 16 hours/day. At 30 mg QD and stead-state 11.5 hours post-dose, Cₚ~4 ng/mL Duration above EC₅₀: 11.5 hours/day.

TABLE 8

| | | | | | | | | | Geometric |
|---|---|---|---|---|---|---|---|---|---|
| | | | | | | | | Geometric | Mean |
| | | | Geometric | Geometric | Geometric | Geometric | Geometric | Mean | Accumulation |
| | | | Mean | Mean | Mean | Mean | Mean | Accumulation | Ratio - |
| | | Dose | HL_Lambda_z | Cmax | Tmax | AUC_0-12 | AUCINF_obs | Ratio - Cmax | AUCtau |
| Cohort | Day_Nominal | (mg) | (h) | (ng/mL) | (h) | (h*ng/mL) | (h*ng/mL) | (Day 15/Day 1) | (Day 15/Day 1) |
| 1 | 1 | 5 | 1.51 | 10.8 | 0.60 | 26 | 27 | | |
| | 7 | 5 | 1.50 | 13.0 | 0.77 | 32 | 32 | | |
| | 15 | 5 | 1.65 | 12.5 | 0.77 | 32 | 32 | 1.2 | 1.2 |
| 2 | 1 | 15 | 1.53 | 28.8 | 0.81 | 71 | 71 | | |
| | 7 | 15 | 1.66 | 42.5 | 0.77 | 109 | 110 | | |
| | 15 | 15 | 1.65 | 38.9 | 0.96 | 110 | 111 | 1.4 | 1.6 |
| 3a | 1 | 30 QD | 1.90 | 104 | 0.68 | 308 | 312 | | |
| | 7 | 30 QD | 1.87 | 124 | 0.87 | 366 | 375 | | |
| | 15 | 30 QD | 1.93 | 119 | 0.96 | 392 | 400 | 1.1 | 1.3 |
| 4 | 1 | 25 | 1.70 | 64.3 | 0.84 | 173 | 175 | | |
| | 7 | 25 | 1.91 | 89.6 | 0.81 | 260 | 265 | | |
| | 15 | 25 | 1.97 | 81.8 | 0.71 | 240 | 245 | 1.3 | 1.4 |
| 5 | 1 | 40 | 1.78 | 102 | 0.84 | 285 | 288 | | |
| | 7 | 40 | 1.85 | 158 | 0.92 | 480 | 487 | | |
| | 15 | 40 | 2.34 | 138 | 1.05 | 472 | 484 | 1.4 | 1.7 |

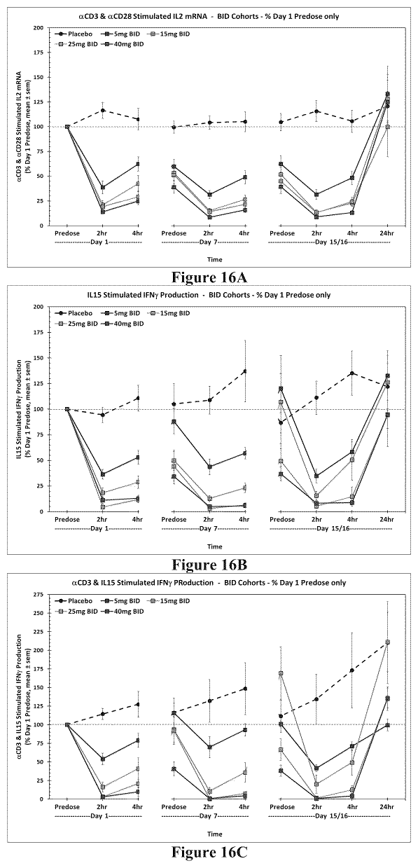

Compound I MAD exploratory pharmacodynamics dose response data for the BID cohorts is shown in FIGS. 16A-C. Compound I shows dose and time dependent modulation of all pharmacodynamic biomarker readouts and 15-40 mg BID doses inhibited biomarker readouts ~50-90% across the dosing intervals.

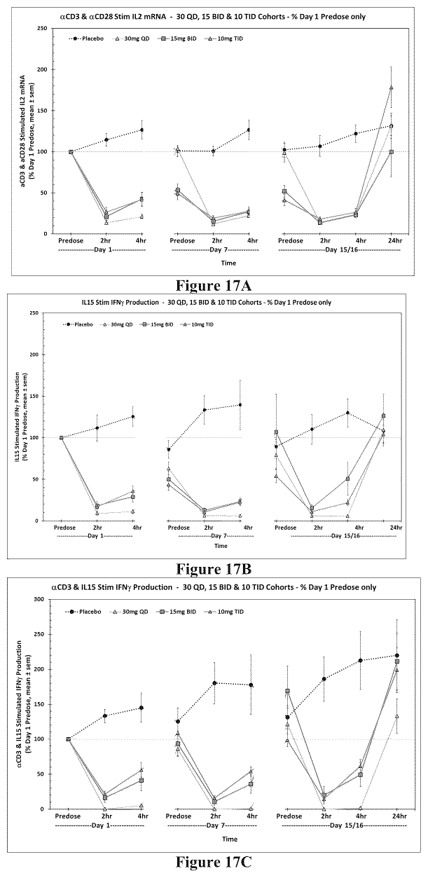

Compound I MAD exploratory pharmacodynamics dose response data for 30 mg QD vs 15 mg BID vs 10 mg TID cohorts is shown in FIGS. 17A-C. 10 mg TID, 15 mg BID, 30 mg BID doses inhibited biomarker readouts to a similar extent.

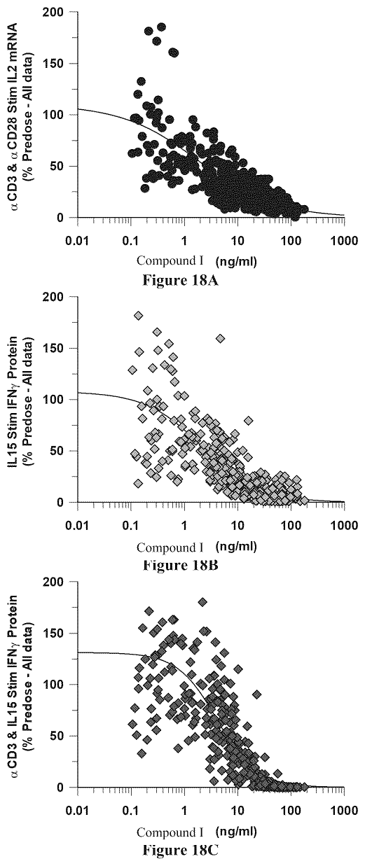

Compound I MAD exploratory pharmacodynamics dose response data for all cohorts is shown in FIGS. 18A-C. EC₅₀ and IC₅₀ data for αCD3 & αCD28 stimulated IL2mRNA, IL15 stimulated IFNγ protein, and αCD3 & IL15 stimulated IFNγ protein is shown in table 9. No significant change when comparing MAD Day 1 EC₅₀ with Days 7 & 15 EC₅₀'s was observed.

TABLE 9

| Target | MAD EC₅₀ | SAD EC₅₀ | Pre-clinical IC₅₀ |
|---|---|---|---|
| αCD3 & αCD28 | 1.9 ng/ml / 5.3 nM | 5.5 ng/ml / 15.2 nM | 7.7 ng/ml / 21.3 nM |
| stimulated IL2mRNA | | | |
| IL15 stimulated | 2.2 ng/ml / 6.1 nM | | 2.9 ng/ml / 8.0 nM |
| IFNγ protein | | | |
| αCD3 & IL15 | 4.1 ng/ml / 11.4 nM | 2.6 ng/ml / 7.3 nM | 3.6 ng/ml / 10.1 nM |
| stimulated | | | |
| IFNγ protein | | | |

Conclusions: Compound I was safe and well tolerated and resulted in dose-dependent increases in plasma Compound I and PD effects consistent with the inhibition of ITK and JAK3.

### Example 3:13 Week Toxicology Study in Rats and Cynomolgus Monkeys

Purpose: Evaluation of toxicity and toxicokinetic (TK) for Compound I, following daily dosing for 13 weeks. The rat study design is shown in table 10.

TABLE 10

| | | Dose | Number of Animals | | | |
|---|---|---|---|---|---|---|
| Group | | Level | Main | | Recovery | |
| No. | Treatment | (mpk) | Male | Female | Males | Female |
| 1 | Control | 0 | 10 | 10 | 5 | 5 |
| 2 | Compound I | 5 | 10 | 10 | 5 | 5 |
| 3 | Compound I | 20 | 10 | 10 | 5 | 5 |
| 4 | Compound I | 80 | 10 | 10 | 5 | 5 |

Group 1 was administered vehicle control article only
Animals were dosed at a volume of 10 mL/kg

Rat Evaluations: No adverse effects observed and no observed adverse effect level at 80 mpk. Margins of safety for the rat study are shown in Tables 11-13.

TABLE 11

| Cₘₐₓ | AUC_{0-24 h} | 15 mg QD in Clinical Trial | | 15 mg BID in Clinical Trial | |
|---|---|---|---|---|---|
| (ng/mL) | (h*ng/mL) | MoS-Cₘₐₓ | MoS-AUC_{0-24 h} | MoS-Cₘₐₓ | MoS-AUC_{0-24 h} |
| 13800 | 45000 | 354 | 408 | 354 | 204 |

At NOAEL of this rat study, Cmax was 13800 ng/mL and AUC0-24 h was 45000 h*ng/mL
Human 15 mg: Cmax = 38.932 ng/mL, AUC0-τ = 110.277 hr*ng/mL
15 mg BID will lead to 2 times of Cmax (around 38.932 ng/mL) daily in human

TABLE 12

| Cₘₐₓ | AUC_{0-24 h} | 25 mg QD in Clinical Trial | | 25 mg BID in Clinical Trial | |
|---|---|---|---|---|---|
| (ng/mL) | (h*ng/mL) | MoS-Cₘₐₓ | MoS-AUC_{0-24 h} | MoS-Cₘₐₓ | MoS-AUC_{0-24 h} |
| 13800 | 45000 | 169 | 187 | 169 | 94 |

At NOAEL of this rat study, Cmax was 13800 ng/mL and AUC0-24 h was 45000 h*ng/mL
Human 25 mg: Cmax = 81.798 ng/mL, AUC0-τ = 240.419 hr*ng/mL
25 mg BID will lead to 2 times of Cmax (around 81.798 ng/mL) daily in human

TABLE 13

| Cₘₐₓ | AUC_{0-24 h} | 30 mg QD in Clinical Trial | | 30 mg BID in Clinical Trial | |
|---|---|---|---|---|---|
| (ng/mL) | (h*ng/mL) | MoS-Cₘₐₓ | MoS-AUC_{0-24 h} | MoS-Cₘₐₓ | MoS-AUC_{0-24 h} |
| 13800 | 45000 | 116 | 115 | 116 | 57 |

At NOAEL of this rat study, Cmax was 13800 ng/mL and AUC0-24 h was 45000 h*ng/mL
Human 30 mg: Cmax = 119 ng/mL, AUC0-τ = 392 hr*ng/mL
30 mg BID will lead to 2 times of Cmax (around 119 ng/mL) daily in human

The cynomolgus monkey study design is shown in table 14.

TABLE 14

| | | Dose | Number of Animals | | | |
|---|---|---|---|---|---|---|
| Group | | Level | Main | | Recovery | |
| No. | Treatment | (mpk) | Male | Female | Male | Female |
| 1 | Control | 0 | 3 | 3 | 2 | 2 |
| 2 | Compound I | 3 | 3 | 3 | 2 | 2 |
| 3 | Compound I | 6 | 3 | 3 | 2 | 2 |
| 4 | Compound I | 12 | 3 | 3 | 2 | 2 |

Group 1 was administered vehicle control article only
Animals were dosed at a volume of 2 mL/kg
One female of 12 mpk group sacrificed on Day 72

Cynomolgus Monkey Evaluations: No adverse effects observed on body weight (overall) and food consumption (overall), clinical pathology in general, anatomic pathology and ophthalmology. Adverse effects included Liquid and non-formed feces associated with dehydration (more significant and persistent in mid/high dose groups), clinical pathology finding, transient but significant body weight loss during this period; dose-dependent opportunistic infection in GI tract: morbidity and significant symptoms necessitating un-scheduled sacrifice and dosing holiday at 12 mpk. The incidents and severity of these effects generally dose-dependent manner and were mild/delayed in the low dose group, moderate/significant in mid/high dose groups. No observed adverse effect level at 3 mpk. Margins of safety for the cynomolgus monkey study are shown in Tables 15-17.

TABLE 15

| Cₘₐₓ | AUC_{0-24 h} | 15 mg QD in Clinical Trial | | 15 mg BID Clinical Trial | |
|---|---|---|---|---|---|
| (ng/mL) | (h*ng/mL) | MoS-Cₘₐₓ | MoS-AUC_{0-24 h} | MoS-Cₘₐₓ | MoS-AUC_{0-24 h} |
| 232 | 356 | 6.0 | 3.2 | 6.0 | 1.6 |

At NOAEL of this monkey study, Cmax was 232 ng/mL and AUC0-24 h was 356 h*ng/mL
Human 15 mg: Cmax = 38.932 ng/mL, AUC0-τ = 110.277 hr*ng/mL
15 mg BID will lead to 2 times of Cmax (around 38.932 ng/mL) daily in human

TABLE 16

| Cₘₐₓ | AUC_{0-24 h} | 25 mg QD in Clinical Trial | | 25 mg BID in Clinical Trial | |
|---|---|---|---|---|---|
| (ng/mL) | (h*ng/mL) | MoS-Cₘₐₓ | MoS-AUC_{0-24 h} | MoS-Cₘₐₓ | MoS-AUC_{0-24 h} |
| 232 | 356 | 2.8 | 1.5 | 2.8 | 0.7 |

At NOAEL of this monkey study, Cmax was 232 ng/mL and AUC0-24 h was 356 h*ng/mL
Human 25 mg: Cmax = 81.798 ng/mL, AUC0-τ = 240.419 hr*ng/mL
25 mg BID will lead to 2 times of Cmax (around 81.798 ng/mL) daily in human

TABLE 17

| Cₘₐₓ | AUC_{0-24 h} | 30 mg QD in Clinical Trial | | 30 mg BID in Clinical Trial | |
|---|---|---|---|---|---|
| (ng/mL) | (h*ng/mL) | MoS-Cₘₐₓ | MoS-AUC_{0-24 h} | MoS-Cₘₐₓ | MoS-AUC_{0-24 h} |
| 232 | 356 | 1.9 | 0.9 | 1.9 | 0.5 |

At NOAEL of this monkey study, Cmax was 232 ng/mL and AUC0-24 h was 356 h*ng/mL
Human 30 mg: Cmax = 119 ng/mL, AUC0-τ = 392 hr*ng/mL
25 mg BID will lead to 2 times of Cmax (around 81.798 ng/mL) daily in human

A summary of the 13-week cynomolgus monkey study is shown in table 18.

TABLE 18
13-Week Monkey Study TK Summary

| | | Day 1 | | | | | | Ratio over time | |
|---|---|---|---|---|---|---|---|---|---|
| Monkey | | Cmax | | Day 28 | | Day 91 | | (D 91/D 1) | |
| Group | | (ng/mL) | AUC0-24 | Cmax | AUC0-24 | Cmax | AUC0-24 | Cmax | AUC |
| Vehicle | | NA | NA | NA | NA | NA | NA | NA | NA |
| 3 | mpk | 104 ± 48.9 | 153 ± 29.1 | 304 ± 156 | 412 ± 191 | 232 ± 53.7 | 356 ± 86.8 | 2.8 | 2.4 |
| 6 | mpk | 401 ± 153 | 692 ± 223 | 985 ± 475 | 1880 ± 911 | 784 ± 512 | 141 ± 619 | 2.2 | 2.2 |
| 12 | mpk | 975 ± 648 | 2510 ± 1420 | 2160 ± 724 | 6930 ± 3790 | 1960 ± 677 | 6430 ± 3660 | 2.5 | 2.6 |

From 3 to 6 mpk (2-fold in dose increase):
On Day 1, Cmax increased 3.9 and AUC increased 4.5; On Day 28, Cmax increased 3.2 fold and AUC increased 4.6; On Day 91, Cmax increased 3.5 and AUC increased 4.0
From 6 mpk to 12 mpp (2-fold in dose increase)
On Day 1, Cmax increased 2.4 and AUC increased 3.6; On Day 28, Cmax increased 2.2 and AUC increased 3.7; on Day 91, Cmax increased 2.5 and AUC increased 4.6
From 3 mpk to 12 mpp (4-fold in dose increase)
On Day 1, Cmax increased 9.4 and AUC increased 16.4; On Day 28, Cmax increased 7.1 and AUC increased 16.8; On Day 91, Cmax increased 8.4 and AUC increased 18.0
Time-dependent and non-linear kinetic: apparent

### Example 4: Pharmacodynamic Analysis Antigen Induced Arthritis Rat Model

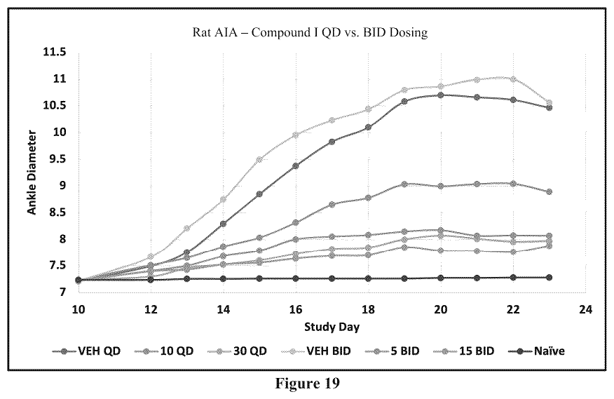

The study evaluated the rat AIA kinetics of ankle diameter swelling. Therapeutic model with PO dosing beginning on day 6. No significant difference between 5 mpk BID, 15 BID and 30 QD. BID dosing is more effective than QD dosing in the rat arthritis model. Results are shown in FIG. 19.

### Example 5: Pharmacodynamic Analysis Comparison of Compound I to Ritlecitinib

Compound I and Ritlecitinib are covalent inhibitors capable of modifying a cysteine residue in ATP site of ITK and JAK3 kinases. Compound I exhibits both ITK and JAK3 inhibition (3-fold selectivity for ITK) while ritlecitinib is a JAK3 selective inhibitor (12-fold selectivity for JAK3) (see tables 19 and 20).

TABLE 19

| | | JAK3 | JAK1/2 |
|---|---|---|---|
| | ITK | hPBMC | hPBMC |
| Cell IC₅₀, nM | (Jurkat/pPLCγ1) | IL-2/pSTAT5 | IFNγ/pSTAT1 |
| Compound I | 8 (81 fold*) | 23 (2.3 fold*) | Inactive |
| Ritlecitinib | 652 | 54 | Inactive |

*potency relative to Ritlecitinib

TABLE 20

| Human | | | | |
|---|---|---|---|---|
| Whole | | IL- | | αCD3 + |
| Blood | αCD3/28/IL-2 | 15/IFN_{γ} | IL-2/pSTAT5 | IL-15/IFN_{γ} |
| EC₅₀, nM | mRNA (2 hr) | (5 hr) | (20 min) | (18 hr) |
| Compound | 21^{c} | 8^{c} | 136^{c} | 10^{c} |
| I | (35 fold*) | (6.5 fold*) | (1.2 fold*) | (3.3 fold*) |
| Ritlecitinib | 740^{b} | 52^{b} | 162^{b} | 33^{b} |

*potency relative to Ritlecitinib

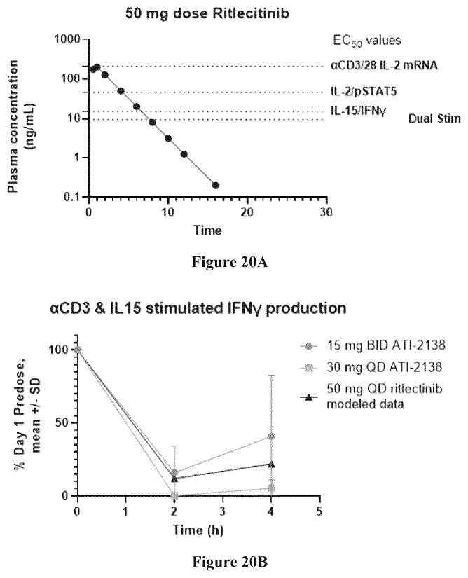

Comparison of Compound I dosing to a 50 mg dose of ritlecitinib (simulated data) is shown in FIGS. 20A-B. Biomarker inhibition with Compound I 15 mg BID and 30 mg QD is similar to that for 50 mg ritlecitinib.

### Example 6: Mouse T Cell Transfer Model of Inflammatory Bowel Disease

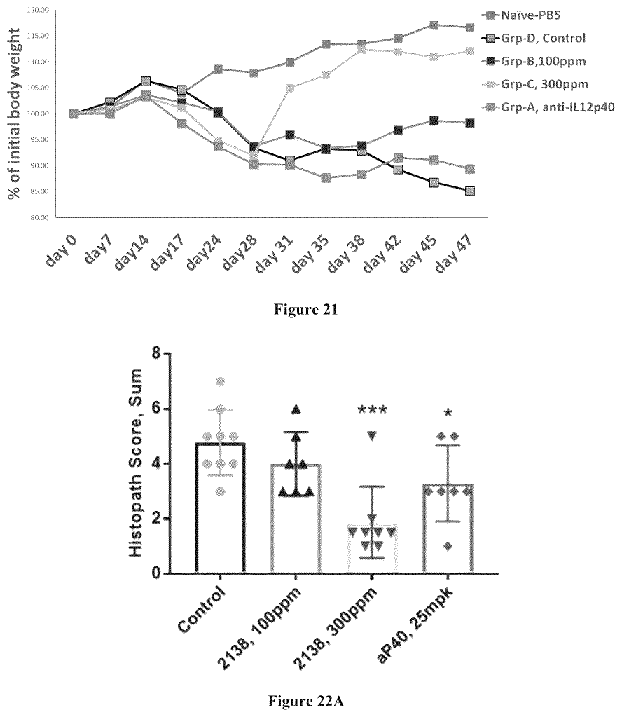

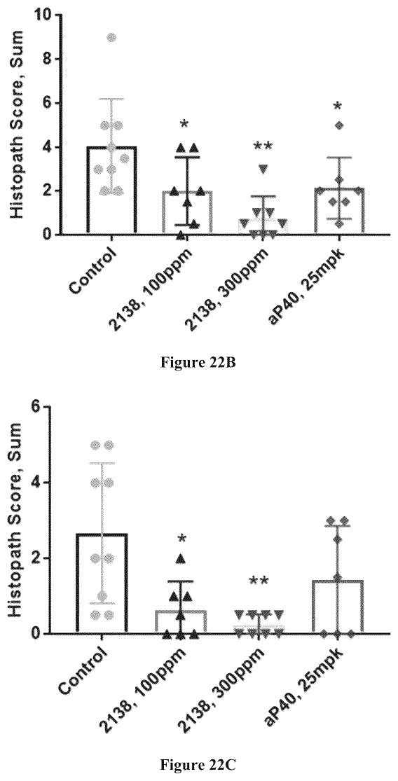

The study evaluated Compound I dose dependent ability to inhibit inflammation in the mouse T cell transfer model of inflammatory bowel disease. Compound I inhibited colitis in the mouse T cell transfer model and dose dependently decreased inflammation in proximal and distal colon, and ileum. The effect was greater than anti-IL12 (P40) that significantly decreased inflammation in the proximal and distal colon. Results are shown in FIGS. 21 and 22A-C.

### Example 7: Compound I UC for Ph2a/POC Trial

Three related molecules have been approved for UC or have positive Phase 2/3 data:

Tofacitinib: Pan-JAKi approved with black-box warning for CV risks

Ritlecitinib (PF-06651600): Pfizer covalent JAK3/ITK with Ph 2 efficacy in UC

Cyclosporine A (CsA) effective/approved for UC—rarely used due to nephrotoxicity

Compound I differs from all these drugs in important ways (key cellular data below):

Unlike tofacitinib, Compound I is completely specific for JAK3, thus no issues with inhibiting other JAK kinases, like JAK2 which can lead to anemia. Compound I's is ~100× more potent on ITK than ritlecitinib. Compound I and CsA are equipotent on T cell receptor blockade, but Compound I toxicity studies show no evidence of nephrotoxicity.

TABLE 21

| Cell IC₅₀, nM | ITK | JAK3 | JAK2 |
|---|---|---|---|
| Compound I | 8 | 23 | Inactive |
| Ritlecitinib | 652 | 54 | Inactive |
| Tofacitinib | Inactive | 11 | 205 |
| CsA | 30 | Inactive | Inactive |

Potential Endpoints:
Clinical Remission—defined as a modified Mayo score of 0-2; Clinical response—decrease from baseline in the modified Mayo Score (mMS)>=2 points and at least 30% better than entry.
Corticosteroid-free remission
endoscopic improvement
endoscopic remission
histologic response/remission

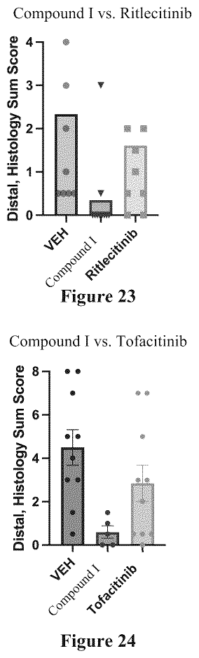

Aclaris experimental colitis data—Histological damage was evaluated for the proximal/distal colon and ileum. Representative data shown here is from the distal colon—across all three regions of GI tissues analyzed. Compound I was more protective than both comparator drugs. Results are shown in FIGS. 23 and 24.

Although embodiments herein have been described in considerable detail with reference to certain preferred embodiments thereof, other versions are possible. Therefore, the spirit and scope of the appended claims should not be limited to the description and the preferred versions contained within this specification.

1. A method for treating an inflammatory condition in a human subject in need thereof, said method comprising:
orally administering to the human subject an oral dose selected from about 10 mg/day to about 80 mg/day of Compound I 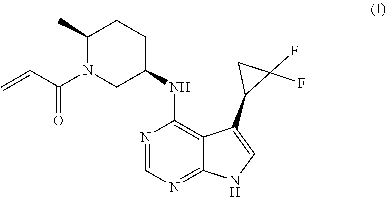 [SMILES: C=CC(=O)N1C[C@H](Nc2ncnc3[nH]cc([C@H]4CC4(F)F)c23)CC[C@@H]1C]
or a derivative thereof to treat said inflammatory condition.

2. The method of claim 1, wherein about 5 mg BID to about 40 mg BID of the Compound I is administered to said subject.

3. The method of claim 1, wherein about 30 mg/day of the Compound I is administered to said subject.

4. The method of claim 1, wherein about 5 mg BID of the Compound I is administered to said subject.

5. The method of claim 1, wherein about 15 mg BID of the Compound I is administered to said subject.

6. The method of claim 1, wherein about 25 mg BID of the Compound I is administered to said subject.

7. The method of claim 1, wherein the inflammatory condition is selected from the group consisting of a chronic inflammatory condition, an acute inflammatory condition, an immuno-inflammatory condition, an autoimmune inflammatory condition, an inflammasomopathy, or a combination thereof.

8. The method of claim 1, wherein the inflammatory condition is selected from the group consisting of atopic dermatitis, palmoplantar pustulosis, fibrosing interstitial lung disease, peripheral T-cell lymphoma, Celiac disease, asthma, recurrent pericarditis, VEXAS syndrome, ulcerative colitis, acute severe ulcerative colitis, Crohn's disease, inflammatory bowel disease, rheumatoid arthritis, psoriatic arthritis, ankylosing spondylitis, acute inflammatory psoriasis, genital psoriasis, autoimmune hepatitis, systemic lupus erythematosus (SLE), organ transplant rejection, chronic graft versus host disease (cGvHD), primary sclerosing cholangitis, or a combination thereof.

9. The method of claim 8, wherein the inflammatory condition is recurrent pericarditis.

10. The method of claim 8, wherein the inflammatory condition is VEXAS syndrome.

11. The method of claim 8, wherein the inflammatory condition is ulcerative colitis.

12. The method of claim 8, wherein the inflammatory condition is rheumatoid arthritis.

13. The method of claim 8, wherein the inflammatory condition is inflammatory bowel disease.

14. The method of claim 13, wherein inflammation resulting from inflammatory bowel disease is in the proximal portion of the bowel.

15. The method of claim 13, wherein inflammation resulting from inflammatory bowel disease is in the distal portion of the bowel.

16. The method of claim 13, wherein inflammation resulting from inflammatory bowel disease is in the ileum portion of the bowel.

17. The method of claim 13, wherein inflammation resulting from inflammatory bowel disease is in the entirety of the bowel.
